(19) United States
(12) Patent Application Publication
(10) Pub. No.: US 2025/0268900 A1
(43) Pub. Date: Aug. 28, 2025

(54) COMBINATION THERAPY USING A SUBSTITUTED PYRIMIDIN-4(3H)-ONE AND NIVOLUMAB AS WELL AS ITS USE IN THE TREATMENT OF CANCER

(71) Applicants: BRISTOL-MYERS SQUIBB COMPANY, Princeton, NJ (US); NAVIRE PHARMA, INC., Palo Alto, CA (US)

(72) Inventors: Lixian JIN, Princeton, NJ (US); Paul HALUSKA, Princeton, NJ (US); Joseph FIORE, Princeton, NJ (US); Lata JAYARAMAN, Cambridge, MA (US); Matthew MEYER, Cambridge, NJ (US); Yu LIU, Cambridge, MA (US); Brian A. POIRSON, Cambridge, MA (US); Nicole PHAN, Cambridge, MA (US); Pedro BELTRAN, Palo Alto, CA (US); Carl DAMBKOWSKI, Palo Alto, CA (US); Justin LIM, Palo Alto, CA (US); Anna WADE, Palo Alto, CA (US); Eli WALLACE, Palo Alto, CA (US); Yuting SUN, Austin, TX (US); Nancy KOHL, Palo Alto, CA (US)

(21) Appl. No.: 18/857,954

(22) PCT Filed: Apr. 21, 2023
(86) PCT No.: PCT/US2023/066088

§ 371 Date: Oct. 18, 2024

Related U.S. Application Data
(60) Provisional application No. 63/334,008, filed on Apr. 22, 2022.

(51) Int. Cl. A61K 31/513; A61K 39/00; A61P 35/00; C07K 16/28

(52) U.S. Cl. CPC A61K 31/513; A61P 35/00; C07K 16/2818; A61K 2039/505; A61K 2039/545

(57) ABSTRACT

The present disclosure provides a method of treating cancer in a subject. The method including administering to the subject: a) a therapeutically effective amount of a compound of formula (I); and b) a therapeutically effective amount of nivolumab, wherein the compound of formula (I) is represented by formula (I) or a pharmaceutically acceptable salt, hydrate, solvate, stereoisomer, conformational isomer, tautomer, or a combination thereof. In particular, the present disclosure provides a method of treating a solid tumor (e.g., an advanced non-small cell lung cancer) with a therapeutically effective amount of a compound of formula (10b) (i.e., 6-((3S,4S)-4-amino-3-methyl-2-oxa-8-azaspiro[4.5]decan-8-yl)-3-(Ra)-(2,3-dichlorophenyl)-2,5-dimethyl-4(3/7)-pyrimidinone) in combination with nivolumab in a subject, wherein the subject has one or more mutations in KRAS.

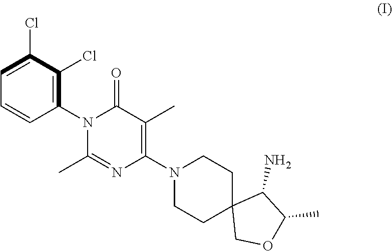

### CROSS-REFERENCES TO RELATED APPLICATIONS

This application claims priority to U.S. Provisional Application No. 63/334,008 filed Apr. 22, 2022, which is incorporated herein in its entirety for all purposes.

### BACKGROUND

Programmed Death-1 (PD-1) is a key immune checkpoint receptor expressed by activated T and B cells and mediates immunosuppression. PD-1 has two ligands, PD-L1 and PD-L2, which are members of the B7 family. PD-L1 is expressed on antigen-presenting cells as well as many human cancers and has been shown to downregulate T cell activation and cytokine secretion upon binding to PD-1. Inhibition of the PD-1/PD-L1 interaction mediates potent antitumor activity in preclinical models and multiple antibody inhibitors of the PD-1/PD-L1 interaction for treating cancer have been approved for clinical use. Among them, pembrolizumab, which targets PD-1 receptors, has been suggested for treatment of a variety of indications including melanoma, lung cancers, head and neck cancer, Hodgkin lymphoma, and stomach cancer. In particular, pembrolizumab is used to treat inoperable or metastatic melanoma and metastatic non-small cell lung cancer (NSCLC); as a first-line treatment for metastatic bladder cancer in patients who can't receive cisplatin-based chemotherapy and have high levels of PD-L1; as a second-line treatment for head and neck squamous cell carcinoma (HNSCC), after platinum-based chemotherapy; for the treatment of adult and pediatric patients with refractory classic Hodgkin's lymphoma (cHL); and for recurrent locally advanced or metastatic esophageal squamous cell carcinoma. Pembrolizumab is also approved for use in treating any unresectable or metastatic solid tumor with certain genetic anomalies (mismatch repair deficiency or high microsatellite instability, and high tumor mutational burden).

Protein-tyrosine phosphatase non-receptor type 11 (PTPN11, also known as Src Homology-2 phosphatase (SHP2)) is a non-receptor protein tyrosine phosphatase encoded by the PTPN11 gene. SHP2 plays a key role in the RTK-mediated MAPK signal transduction pathway. This PTP contains two tandem Src homology-2 (SH2) domains, which function as phospho-tyrosine binding domains, a catalytic domain, and a C-terminal tail. In the basal state the protein typically exists in an inactive, self-inhibited conformation with the N-terminal SH2 domain blocking the active site. When stimulated by signal transduction mediated by cytokines and growth factor binding of phosphorylated proteins to the SH2 domains the auto-inhibition is relieved, this makes the active site available for dephosphorylation of PTPN11 substrates (MG Mohl, BG Neel, Curr. Opin. Genetics Dev. 2007, 17, 23-30. KS Grossmann, Adv. Cancer Res. 2010, 106, 53-89. W. Q. Huang et. al. Curr. Cancer Drug Targets 2014, 14, 567-588. C. Gordon et. al. Cancer Metastasis Rev. 2008, 27, 179-192.).

Germ-line and somatic mutations in PTPN11 have been reported in several human diseases resulting in gain-of-function in the catalytic activity, including Noonan Syndrome and Leopard Syndrome; as well as multiple cancers such as juvenile myelomonocytic leukemia, neuroblastoma, myelodysplastic syndrome, B cell acute lymphoblastic leukemia/lymphoma, melanoma, acute myeloid leukemia and cancers of the breast, lung and colon (MG Mohl, BG Neel, Curr. Opin. Genetics Dev. 2007, 17, 23-30). Recent studies have demonstrated that single PTPN11 mutations are able to induce Noonan syndrome, JMML-like myeloproliferative disease and acute leukemia in mice. These mutations disrupt the auto-inhibition between the N—SH2 domains and the catalytic site allowing constitutive access of substrates to the catalytic site of the enzyme (E. Darian et al, Proteins, 2011, 79, 1573-1588. Z-H Yu et al, JBC, 2013, 288, 10472, W Qiu et al BMC Struct. Biol. 2014, 14, 10).

PTPN11 is widely expressed in most tissues and plays a regulatory role in various cell signaling events that are important for a diversity of cell functions that includes proliferation, differentiation, cell cycle maintenance, epithelial-mesenchymal transition (EMT), mitogenic activation, metabolic control, transcription regulation, and cell migration, through multiple signaling pathways including the Ras-MAPK, the JAK-STAT or the PI3K-AKT pathways (Tajan, M. et. al. Eur. J. Medical Genetics, 2015, 58, 509-525. Prahallad, A. et. al. Cell Reports, 2015, 12, 1978-1985).

Additionally there is growing evidence that PTPN11/SHP2 is implicated in immune evasion during tumorigenesis, and hence a SHP2 inhibitor could stimulate the immune response in cancer patients (Cancer Res. 2015 Feb. 1; 75(3):508-18. T Yokosuka T, J Exp Med. 2012, 209(6), 1201. S Amarnath Sci Transl Med. 2011, 3, 111ra120. T Okazaki, PNAS 2001, 98:24, 13866-71).

Substituted pyrimin-4(3H)-one compounds refer to a class of compounds having inhibitory activities against PTPN11/SHP2, as disclosed in International Patent Application No. PCT/US2019/045903 filed Aug. 9, 2019, and represented by the following formula:

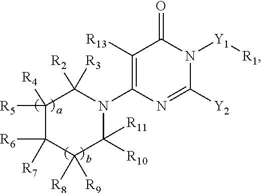

or a pharmaceutically acceptable salt, hydrate, solvate, stereoisomer, conformational isomer, tautomer, or a combination thereof, wherein the subscripts a and b, Y₁, Y₂, and R₁, R₂, R₃, R₄, R₅, R₆, R₇, R₈, R₉, R₁₀, R₁₁, and R₁₃ are as provided in PCT/US2019/045903, which is incorporated herein in its entirety for all purposes. In particular, the substituted pyrimin-4(3H)-one compound is represented by formula (I):

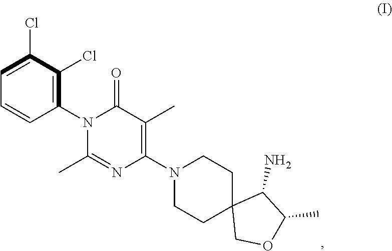

or a pharmaceutically acceptable salt, hydrate, solvate, stereoisomer, conformational isomer, tautomer, or a combination thereof. In certain embodiments, the compound of formula (I) is Compound (10b), represented by the formula:

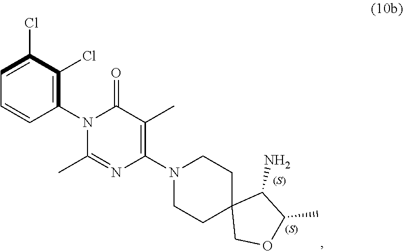

having the name of 6-((3S,4S)-4-amino-3-methyl-2-oxa-8-azaspiro[4.5]decan-8-yl)-3-(Rₐ)-(2,3-dichlorophenyl)-2,5-dimethyl-4(3H)-pyrimidinone.

The compound of formula (I), in particular formula (10b), is a potent, selective, orally active allosteric inhibitor of Src Homology-2 Phosphatase (SHP2) (also known as protein tyrosine phosphatase non-receptor type 11 (PTPN11)), a tyrosine phosphatase that plays a key role in the receptor tyrosine kinase (RTK)-mediated mitogen activated protein kinase (MAPK) signal transduction pathway (Matozaki, 2009). Key components of the MAPK pathway include the small GTPase RAS, the serine/threonine-protein kinase RAF, mitogen-activated protein kinase kinase (MEK) and extracellular signal activated kinase (ERK). In cells, SHP2 binds to phosphorylated tyrosine residues in the intracellular domain of RTKs such as the Epidermal Growth Factor Receptor (EGFR), leading to activation of the downstream MAPK signaling pathway.

However, in spite of the treatment options with PD-1/PD-L1 inhibitors, cases of resistance occur. Thus, there remains a need for effective and safe therapeutic agents to treat such cases of resistance.

### BRIEF SUMMARY

The present disclosure provides methods for treating various cancers, the method including administration a PD-1/PD-L1 inhibitor (e.g., nivolumab) and a PTPN11 inhibitor (e.g., a compound of formula (I) or (10b)). The combination therapies provided herein are useful in the treatment of cancers including lung cancers (e.g., non-small cell lung cancer).

In one aspect, the present disclosure provides a method of treating cancer (e.g., an advanced non-small cell lung cancer) in a subject, the method including administering to a subject in need thereof:
a) a therapeutically effective amount of a compound represented by formula (I):

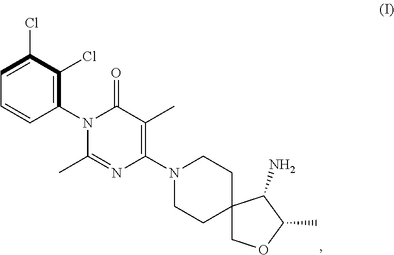

or a pharmaceutically acceptable salt, hydrate, solvate, stereoisomer, conformational isomer, tautomer, or a combination thereof; and
b) a therapeutically effective amount of nivolumab.

In another aspect, the present disclosure provides a kit for treating cancer in a subject, the kit including:
a) a therapeutically effective amount of a compound represented by formula (I); and
b) a therapeutically effective amount of nivolumab, together with instruction for effective administration,
   wherein the compound of formula (I) is as defined and described herein.

In some embodiments, the compound of formula (I) is represented by formula (10b):

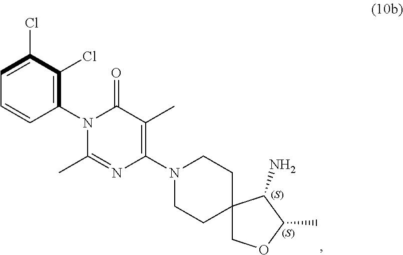

having the name of 6-((3S,4S)-4-amino-3-methyl-2-oxa-8-azaspiro[4.5]decan-8-yl)-3-(Rₐ)-(2,3-dichlorophenyl)-2,5-dimethyl-4(3H)-pyrimidinone.

### BRIEF DESCRIPTION OF THE DRAWINGS

FIG. 1A-FIG. 1B show results from a study of the effect of Compound (10b) on CD107a expression of CD8+ T cells. FIG. 1A: Donors 1 and 3; and FIG. 1B: Donors 2 and 4.

FIG. 2 shows an overall design of A Phase 1 study of Compound (10b) in combination with nivolumab in patients with advanced non-small cell lung cancer with a KRAS mutation.

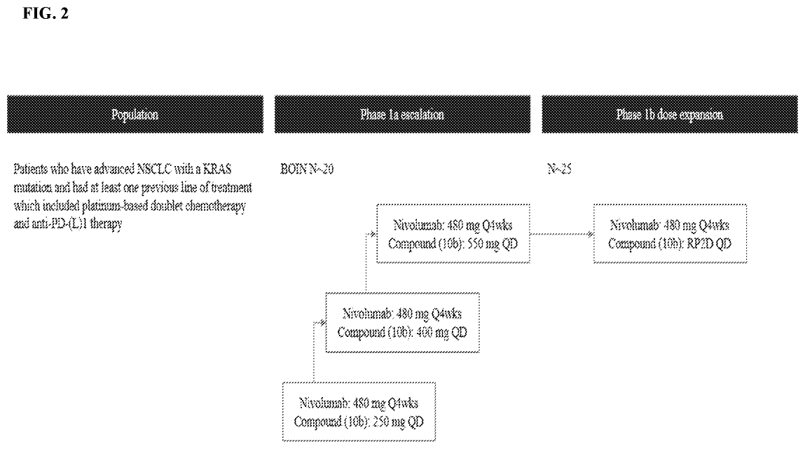

FIG. 3 shows a flowchart for a trial conducted using the BOIN Design. Abbreviations: BOIN=Bayesian optimal interval design; DLT=dose limiting toxicity; MTD=maximum tolerated dose. Note: λe=19.7% and λd=29.8%. In practice, with 6 patients/cohort, if the DLT rate is ≤⅙ then escalate the dose, if the DLT rate is ≥ 2/6 then de-escalate the dose.

FIG. 4 shows progression-free to tumor burden endpoint after CT-26 KRAS D12C KI tumor-bearing female Balb/c mice were treated with formula (10b) and ANTIBODY A (as an anti-PD-1 therapy), alone or in combination.

FIG. 5 shows a statistically significant delay in tumor progression to burden, when CT-26 KRAS D12C KI tumor-bearing female Balb/c mice were treated with a combination of formula (10b) and ANTIBODY A.

FIG. 6 shows tumor volumes after CT-26 KRAS D12C KI tumor-bearing female Balb/c mice were treated with formula (10b) and ANTIBODY A (as an anti-PD-1 therapy), alone or in combination.

### DETAILED DESCRIPTION

### I. General

The present disclosure provides a combination therapy method of treating cancer (e.g., a solid tumor) in a subject. The method includes administering to the subject a) a therapeutically effective amount of a compound of formula (I) (as a PTPN11 inhibitor); and b) a therapeutically effective amount of nivolumab, wherein the compound of formula (I) is as defined and described herein. In particular, the cancer is characterized by a KRAS mutation, such as a KRAS G12 mutation other than a Q61X mutation. In some instances, the cancer is a solid tumor, such as an advanced non-small cell lung cancer (NSCLC). Also provided are a kit including the compound and nivolumab for treating cancer in a subject.

### II. Definitions

As used herein, the terms below have the meanings indicated.

Unless specifically indicated otherwise, the group

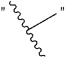

as used herein in any one of formulae (e.g., (I), (Ia), and (10b)), refers to methyl.

“Comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with 12 water molecule or from 1 to 10 water molecules.

Asymmetric centers exist in the compounds disclosed herein. These centers are designated by the symbols “R” or “S,” depending on the configuration of substituents around the chiral carbon atom. It should be understood that the present disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method. Starting compounds of particular stereochemistry are either commercially available or can be made and resolved by various techniques. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers include keto/enol, enamine/imine, and lactam/lactim tautomers. Additional examples of tautomers include 2-hydroxypyridine/2(1H)-pyridone and 2-aminopyridine/2(1H)-iminopyridone tautomers.

Conformational isomers exist in the compounds disclosed herein. When R₁ is aryl or heteroaryl in the formula:

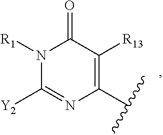

the aryl or heteroaryl group can orient in different conformations in relation to the pyrimidinone moiety, as represented by:

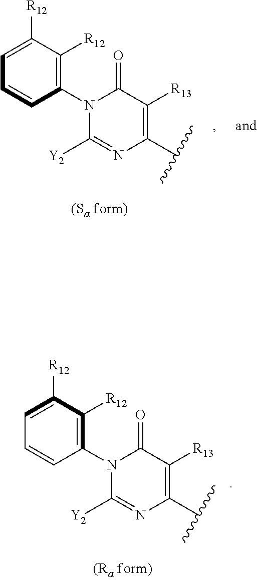

These forms are designated by the symbols “Sₐ” or “Rₐ”, depending on the conformation of the aryl or heteroaryl group in relation to the pyrimidinone moiety. Examples of “Sₐ” and “Rₐ” forms can be found in Examples 1-20 of International Patent Application No. PCT/US2019/045903, which is incorporated herein in its entirety for all purposes. The compound of formula (10b) is substantially in a “Rₐ” form.

“Pharmaceutically acceptable” refers to those compounds (salts, hydrates, solvates, stereoisomers, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

“Combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PTPN11 inhibitor” is used herein to refer to a compound that exhibits an IC₅₀ with respect to PTPN11 activity of no more than about 100 micromolar (M) and more typically not more than about 50 μM, as measured in the PTPN11 assay described generally in International Patent Application No. PCT/US2019/045903 (e.g., the enzymatic activity of recombinant human PTPN11 proteins of Example 21). “IC₅₀” is that concentration of inhibitor which reduces the activity of an enzyme (e.g., PTPN11) to half-maximal level. In certain embodiments, compounds disclosed in PCT/US2019/045903 exhibit an IC₅₀ of no more than about 10 μM for inhibition of PTPN11; in further embodiments, compounds exhibit an IC₅₀ of no more than about 1 μM for inhibition of PTPN11; in yet further embodiments, compounds will exhibit an IC₅₀ of not more than about 200 nM for inhibition of PTPN11; in yet further embodiments, compounds will exhibit an IC₅₀ of not more than about 100 nM for inhibition of PTPN11; and in yet further embodiments, compounds will exhibit an IC₅₀ of not more than about 50 nM for inhibition of PTPN11, as measured in the PTPN11 assay described therein. In certain embodiments, the compound of formula (I) or (10b) exhibits an IC₅₀ of no more than 50 nM for inhibition of PTPN11 (e.g., a PTPN11-E76K mutant enzyme).

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating”, and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters; including the results of a physical examination, assay (e.g., analysis of a fluid of a subject, such as blood, plasma, or urine), imaging analysis, neuropsychiatric exams, and/or a psychiatric evaluation.

“Administering” refers to therapeutic provision of the compound or a form thereof to a subject, such as by oral administration or intravenous administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age).

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors. Other pharmaceutical excipients can be useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include the compound of formula (I) or (10b) and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, the percent weights reported herein are of the uncoated tablet.

Unless specifically indicated otherwise, the content of the compound of formula (I) or (10b) in, e.g., a tablet formulation is calculated based on the normalized weight of the compound of formula (I) or (10b) on a salt-free and anhydrous basis. That is, the salt and/or water content in the compound of formula (I) or (10b) is not included in the calculation.

“PD-1/PD-L1 inhibitor” (also known as immune checkpoint inhibitors) as used herein refers to a compound which targets, decreases, or inhibits the synthesis or biological activity of programmed cell death protein 1 (PD-1) and/or programmed death-ligand 1 (PD-L1). PD-1 has two ligands, PD-L1 and PD-L2. The PD-1/PD-L1 inhibitor blocks PD-L1 and/or PD-L2 binding to PD-1. The PD-1/PD-L1 inhibitor may at least partially inhibit PD-1 and/or PD-L1. The PD-1/PD-L1 inhibitor may be a PD-1 inhibitor. The PD-1/PD-L1 inhibitor may be a PD-L1 inhibitor. The PD-1/PD-L1 inhibitor may be a selective PD-1/PD-L1 inhibitor. In those cases, the selective PD-1/PD-L1 inhibitor may have high potency for PD-1/PD-L1, along with low affinity for related programmed death-ligand 2 (PD-L2). Examples of PD-1/PD-L1 inhibitors include pembrolizumab, nivolumab, atezolizumab, durvalumab, avelumab, cemiplimab-rwlc, camrelizumab, toripalimab, sintilimab, prolgolimab, tislelizumab, balstilimab, dostarlimab, M7824, spartalizumab, sasanlimab, retifanlimab, BMS-986213, and tebotelimab.

“PD-L1-positive cancer” refers to a cancer with expression or overexpression of PD-L1.

“A cancer resistant to a PD-1/PD-L1 inhibitor”, “a cancer that is a PD-1/PD-L1-positive cancer resistant to a PD-1/PD-L1 inhibitor”, and/or “a cancer that is a KRAS-positive cancer resistant to a PD-1/PD-L1 inhibitor” refer to a cancer or tumor that either fails to respond favorably to treatment with a prior PD-1/PD-L1 inhibitor, or alternatively, recurs or relapses after responding favorably to a PD-1/PD-L1 inhibitor. In some embodiments, a cancer is resistant to nivolumab.

“KRAS-positive cancer” refers to a cancer with the KRAS gene rearranged, mutated, or amplified. “KRAS G12C-positive cancer” refers to a cancer with the KRAS G12C gene rearranged, mutated, or amplified.

“A cancer resistant to a KRAS inhibitor” and/or “a cancer that is a KRAS-positive cancer resistant to a KRAS inhibitor” refer to a cancer or tumor that either fails to respond favorably to treatment with a prior KRAS inhibitor, or alternatively, recurs or relapses after responding favorably to a KRAS inhibitor. “A cancer resistant to a KRAS G12C inhibitor” and/or “a cancer that is a KRAS G12C-positive cancer resistant to a KRAS G12C inhibitor” refer to a cancer or tumor that either fails to respond favorably to treatment with a prior KRAS G12C inhibitor, or alternatively, recurs or relapses after responding favorably to a KRAS G12C inhibitor.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a PTPN11 inhibitor as defined herein; and a PD-1/PD-L1 inhibitor as defined herein, which is greater than the simple addition of the effects of each drug administered by themselves. The effect can be, for example, slowing the symptomatic progression of a proliferative disease, such as cancer, particularly lung cancer (e.g., non-small cell lung cancer), or symptoms thereof. Analogously, a “synergistically effective amount” refers to the amount needed to obtain a synergistic effect.

“A,” “an,” or “a(n)”, when used in reference to a group of substituents or “substituent group” herein, mean at least one. For example, where a compound is substituted with “an” alkyl or aryl, the compound is substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Combination Therapy

In one aspect, the present disclosure provides a method of treating cancer in a subject. The method includes administering to the subject:
a) a therapeutically effective amount of a compound represented by formula (I):

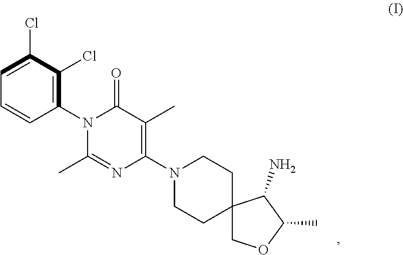

or a pharmaceutically acceptable salt, hydrate, solvate, stereoisomer, conformational isomer, tautomer, or a combination thereof; and
b) a therapeutically effective amount of nivolumab.

### III-1: Compound of Formula (I)

The compound of formula (I) can be in a pharmaceutically acceptable salt form or in a neutral form, each of which is optionally in a solvate or a hydrate form.

In some embodiments, the compound of formula (I) is in a pharmaceutically acceptable salt form. In some embodiments, a pharmaceutically acceptable acid addition salt of the compound of formula (I) is represented by formula (Ia):

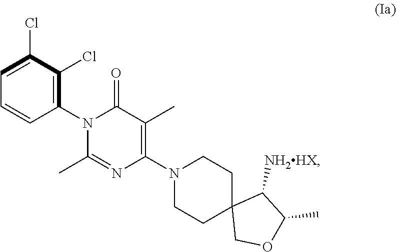

wherein HX is a pharmaceutically acceptable acid addition.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

In some embodiments, the compound of formula (I) is in a neutral form.

In some embodiments, the compound of formula (I) has a substantially moiety of 6-((3S,4S)-4-amino-3-methyl-2-oxa-8-azaspiro[4.5]decan-8-yl) with stereochemistry as shown in formula (10b):

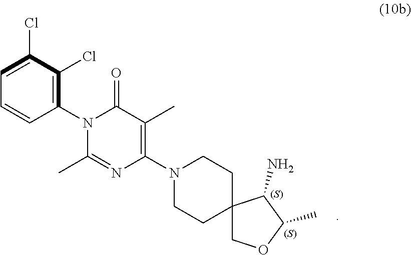

In some embodiments, the compound of formula (I) is substantially in a Rₐ conformation as shown in formula (10b):

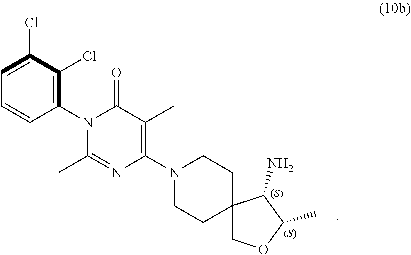

In some embodiments, the compound of formula (I) is represented by formula (10b):

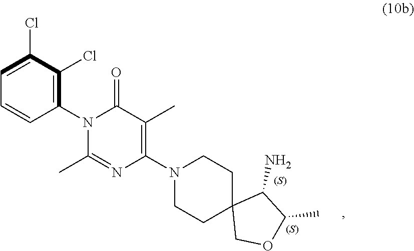

having the name of 6-((3S,4S)-4-amino-3-methyl-2-oxa-8-azaspiro[4.5]decan-8-yl)-3-(Rₐ)-(2,3-dichlorophenyl)-2,5-dimethyl-4(3H)-pyrimidinone.

In some embodiments, the compound of formula (I) or (10b) is in a neutral form.

In some embodiments, the compound of formula (I) includes one or more corresponding enantiomer, diastereomers, and/or conformational isomers of the compound of formula (10b), as represented by formulae, respectively:

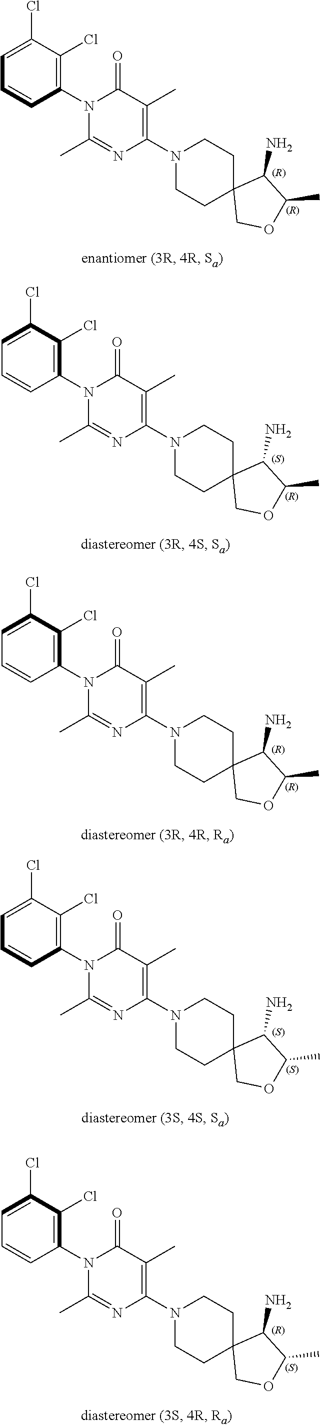

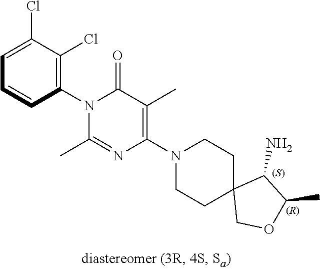

In some embodiments, the compound of formula (10b) has a purity of at least about 95 area % determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, determined by a chiral high-performance liquid chromatography (HPLC). In some embodiments, the compound of formula (10b) has a purity of from about 98 area % to about 99 area %.

In some embodiments, the compound of formula (1) includes one or more corresponding enantiomer, diastereomers, and/or conformational isomers of the compound of formula (10b), as represented by the formulae above; and a total of the one or more isomers is no more than about 5 area % determined by a chiral high-performance liquid chromatography (HPLC).

In some embodiments, the corresponding enantiomer, diastereomers, and/or conformational isomers of the compound of formula (10b) are present in the compound of formula (I) meet acceptance criteria as follows: enantiomer (3R, 4R, Sₐ)≤0.5 area %; diastereomer (3R, 4S, Rₐ)≤1.2 area %; diastereomer (3S, 4R, Sₐ)≤0.5 area %; diastereomer (3R, 4R, Rₐ)≤0.5 area %; diastereomer (3S, 4S, Sₐ)≤0.5 area %; diastereomer (3S, 4R, Rₐ)≤0.5 area %; and diastereomer (3R, 4S, Sₐ)≤0.5 area %, each of which is determined by a chiral high-performance liquid chromatography (HPLC). In some embodiments, the compound of formula (10b) has a purity of at least about 95 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 98 area % to about 99 area %, wherein enantiomer (3R, 4R, Sₐ) is not detected; diastereomer (3R, 4S, Rₐ) is about 0.86 area %; diastereomer (3S, 4R, Sₐ) is not detected; diastereomer (3R, 4R, Rₐ) is about 0.07 area %; diastereomer (3S, 4S, Sₐ) is not detected; diastereomer (3S, 4R, Rₐ) is not detected; and diastereomer (3R, 4S, Sₐ) is not detected, each of which is determined by a chiral high-performance liquid chromatography (HPLC).

In some embodiments, the compound of any one of formulae (I), (Ia), and (10b) is in a solvate and/or a hydrate form.

### III-2. Cancer/Solid Tumor

The cancer can be any cancer that responds to the treatment of a PTPN11 inhibitor and/or a PD-1/PD-L1 inhibitor. In some embodiments, the cancer expresses PD-L1. In some embodiments, the cancer is characterized by a high level of microsatellite instability (MSI-H), a deficient mismatch repair (dMMR), a high level of tumor mutational burden (TMB-H), or a combination thereof. In some embodiments, the cancer is characterized by a high level of microsatellite instability (MSI-H). In some embodiments, the cancer is characterized by a deficient mismatch repair (dMMR). In some embodiments, the cancer is characterized by a high level of tumor mutational burden (TMB-H). In some embodiments, the cancer is caused and/or characterized by a KRAS mutation, such as a KRAS G12C mutation. In some embodiments, the cancer is characterized by a KRAS mutation other than a Q61X mutation. In some embodiments, the cancer is a PD-L1-positive cancer. In some embodiments, the cancer is a KRAS-positive cancer. In some embodiments, the cancer is a KRAS G12C-positive cancer (e.g., a cancer characterized by a G12C mutation in KRAS).

In some embodiments, the cancer is characterized by a KRAS mutation, such as a mutation in codon 12, 13, or 61 (e.g., a G12C, G12D, G12S, G12V, G12R, G12A, G12F, G12I, G12L, G13D, G13A, G13C, G13R, G13S, G13V, Q61E, Q61K, Q61L, Q61P, Q61R, and/or Q61H mutation). In some embodiments, the cancer is characterized by a KRAS mutation selected from a G12C, G12D, G12S, G12V, G12R, G12A, G13D, G13A, G13C, G13R, G13S, G13V, and a combination thereof. In some embodiments, the cancer is characterized by a KRAS G12C mutation. In some embodiments, the KRAS protein does not include a mutation at Q61.

The cancer can be characterized by a solid tumor and/or a liquid tumor. In some embodiments, the cancer includes a solid tumor. In some embodiments, the cancer includes a liquid tumor.

In some embodiments, the cancer is anal cancer, biliary tract cancer, bladder cancer, brain cancer, cervical cancer, colorectal cancer (CRC), endometrial cancer, esophageal cancer, gastric cancer, head and neck squamous cell carcinoma (HNSCC), hepatocellular carcinoma (HCC), merkel cell carcinoma, melanoma, mesothelioma, non-small cell lung cancer (NSCLC), ovarian cancer, prostate cancer, renal cell carcinoma (RCC), small cell lung cancer (SCLC), squamous cell carcinoma (SCC), triple negative breast cancer (TNBC), Hodgkin's lymphoma, primary mediastinal large B-cell lymphoma (PMBCL), diffuse large B-cell lymphoma (DLBCL), or a combination thereof. In some embodiments, the cancer is anal cancer, biliary tract cancer, bladder cancer, brain cancer, cervical cancer, colorectal cancer (CRC), endometrial cancer, esophageal cancer, gastric cancer, head and neck squamous cell carcinoma (HNSCC), hepatocellular carcinoma (HCC), merkel cell carcinoma, melanoma, mesothelioma, non-small cell lung cancer (NSCLC), ovarian cancer, prostate cancer, renal cell carcinoma (RCC), small cell lung cancer (SCLC), squamous cell carcinoma (SCC), triple negative breast cancer (TNBC), or a combination thereof. In some embodiments, the cancer is Hodgkin's lymphoma, primary mediastinal large B-cell lymphoma (PMBCL), diffuse large B-cell lymphoma (DLBCL), or a combination thereof. In some embodiments, the cancer is Hodgkin's lymphoma. In some embodiments, the cancer is diffuse large B-cell lymphoma (DLBCL).

In some embodiments, the cancer is colorectal cancer (CRC). In some embodiments, the cancer is non-small cell lung cancer (NSCLC).

In some embodiments, the cancer is colorectal cancer (CRC) characterized by a KRAS mutation, such as a mutation in codon 12 (e.g., KRAS G12C, G12D, or G12R).

In some embodiments, the cancer is NSCLC characterized by a KRAS mutation, such as a KRAS G12C mutation. In some embodiments, a KRAS protein includes a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein includes a G12C, G12D, or G12V mutation. In some embodiments, the cancer is NSCLC characterized by a G12C, G12D, or G12V mutation in KRAS. In some embodiments, the cancer is NSCLC characterized by a mutation in an epidermal growth factor receptor (EGFR) protein. In some embodiments, the cancer is NSCLC that is not characterized by a mutation in EGFR, KRAS, or anaplastic lymphoma kinase (ALK).

The cancer can also be any cancer that is resistant to the treatment of a PD-1/PD-L1 inhibitor. In some embodiments, the cancer is resistant to a PD-1/PD-L1 inhibitor as defined and described herein. In some embodiments, the cancer is characterized by intrinsic and/or acquired resistance to a PD-1/PD-L1 inhibitor. In some embodiments, the cancer is a PD-1/PD-L1-positive cancer resistant to a PD-1/PD-L1 inhibitor. In some embodiments, the cancer is a PD-1/PD-L1-positive cancer characterized by intrinsic and/or acquired resistance to a PD-1/PD-L1 inhibitor. In some embodiments, the cancer is a KRAS-positive cancer resistant to a PD-1/PD-L1 inhibitor. In some embodiments, the cancer is a KRAS-positive cancer characterized by intrinsic and/or acquired resistance to a PD-1/PD-L1 inhibitor.

In some embodiments, the cancer is resistant to a PD-1/PD-L1 inhibitor selected from the group consisting of pembrolizumab, nivolumab, atezolizumab, durvalumab, avelumab, cemiplimab-rwlc, camrelizumab, toripalimab, sintilimab, prolgolimab, tislelizumab, balstilimab, dostarlimab, M7824, spartalizumab, sasanlimab, retifanlimab, BMS-986213, and tebotelimab. In some embodiments, the cancer is resistant to pembrolizumab. In some embodiments, the cancer is resistant to nivolumab.

In some embodiments, the cancer is a PD-L1-positive cancer resistant to a PD-1/PD-L1 inhibitor selected from the group consisting of pembrolizumab, nivolumab, atezolizumab, durvalumab, avelumab, cemiplimab-rwlc, camrelizumab, toripalimab, sintilimab, prolgolimab, tislelizumab, balstilimab, dostarlimab, M7824, spartalizumab, sasanlimab, retifanlimab, BMS-986213, and tebotelimab. In some embodiments, the cancer is a PD-L1-positive cancer resistant to pembrolizumab. In some embodiments, the cancer is a PD-L1-positive cancer resistant to nivolumab.

In some embodiments, the cancer is a KRAS-positive cancer resistant to a PD-1/PD-L1 inhibitor selected from the group consisting of pembrolizumab, nivolumab, atezolizumab, durvalumab, avelumab, cemiplimab-rwlc, camrelizumab, toripalimab, sintilimab, prolgolimab, tislelizumab, balstilimab, dostarlimab, M7824, spartalizumab, sasanlimab, retifanlimab, BMS-986213, and tebotelimab. In some embodiments, the cancer is a KRAS-positive cancer resistant to pembrolizumab. In some embodiments, the cancer is a KRAS-positive cancer resistant to nivolumab.

In some embodiments, the cancer is characterized by intrinsic and/or acquired resistance to another therapy such as a KRAS modulator, platinum-based therapy, or taxane therapy.

The solid tumor can be any solid tumor that responds to the treatment of a PTPN11 inhibitor and an anti-PD-1/PD-L1 agent (e.g., nivolumab). In some embodiments, the solid tumor is a tumor with one or more genes in KRAS rearranged, mutated, or amplified, provided that the tumor is other than caused by one or more additional activating mutations in BRAF V600X, PTPN11 (SHP2), or KRAS Q61X.

In some embodiments, the solid tumor is an advanced non-small cell lung cancer (NSCLC) caused by a mutation in KRAS. In some embodiments, the solid tumor is an advanced non-small cell lung cancer (NSCLC) caused by a mutation in KRAS, provided that the tumor is other than caused by one or more additional activating mutations in BRAF V600X, PTPN11 (SHP2), or KRAS Q61X. In some embodiments, the solid tumor is a KRAS G12C-positive solid tumor. In some embodiments, the solid tumor is an advanced or metastatic KRAS G12C-positive non-small cell lung cancer (NSCLC).

The solid tumor can also be any tumor that is resistant to the treatment of a KRAS G12C inhibitor (e.g., sotorasib (AMG-510), adagrasib (MRTX-849), MRTX1257, ARS-853, ARS-1620, JNJ-74699157(ARS-3248), LY3537982, and LY3499446). In some embodiments, the solid tumor is resistant to a KRAS G12C inhibitor. In some embodiments, the solid tumor is characterized by intrinsic and/or acquired resistance to a KRAS G12C inhibitor as defined and described herein. In some embodiments, the solid tumor is a KRAS G12C-positive solid tumor resistant to a KRAS G12C inhibitor. In some embodiments, the solid tumor is a KRAS G12C-positive solid tumor characterized by intrinsic and/or acquired resistance to a KRAS G12C inhibitor. In some embodiments, the solid tumor is resistant to the treatment of a KRAS G12C inhibitor selected from the group consisting of sotorasib (AMG-510), adagrasib (MRTX-849), MRTX1257, ARS-853, ARS-1620, JNJ-74699157(ARS-3248), LY3537982, and LY3499446. In some embodiments, the solid tumor is resistant to sotorasib (AMG-510). In some embodiments, the solid tumor is resistant to adagrasib (MRTX-849). In some embodiments, the solid tumor is a KRAS-positive solid tumor resistant to the treatment of a KRAS G12C inhibitor selected from the group consisting of sotorasib (AMG-510), adagrasib (MRTX-849), MRTX1257, ARS-853, ARS-1620, JNJ-74699157(ARS-3248), LY3537982, and LY3499446. In some embodiments, the solid tumor is a KRAS-positive solid tumor resistant to sotorasib (AMG-510). In some embodiments, the solid tumor is a KRAS-positive solid tumor resistant to adagrasib (MRTX-849). In some embodiments, the solid tumor is a KRAS G12C-positive solid tumor resistant to the treatment of a KRAS G12C inhibitor selected from the group consisting of sotorasib (AMG-510), adagrasib (MRTX-849), MRTX1257, ARS-853, ARS-1620, JNJ-74699157(ARS-3248), LY3537982, and LY3499446. In some embodiments, the solid tumor is a KRAS G12C-positive solid tumor resistant to sotorasib (AMG-510). In some embodiments, the solid tumor is a KRAS G12C-positive solid tumor resistant to adagrasib (MRTX-849).

The solid tumor can also be any tumor that is resistant to the treatment of a PD-1/PD-L1 inhibitor (e.g., nivolumab). In some embodiments, the solid tumor is resistant to nivolumab. In some embodiments, the solid tumor is characterized by intrinsic and/or acquired resistance to nivolumab. In some embodiments, the solid tumor is a KRAS-positive solid tumor resistant to nivolumab. In some embodiments, the solid tumor is a KRAS-positive solid tumor characterized by intrinsic and/or acquired resistance to nivolumab.

In any one of embodiments, a standard of care or curative therapy is unavailable for treating cancer or a solid tumor, as described herein.

### III-3: Subject

In some embodiments, the subject is human. In some embodiments, the subject is under the care of a medical practitioner, such as a physician. In some embodiments, the subject has been diagnosed with the cancer. In some embodiments, the subject has relapsed. In some embodiments, the subject has previously entered remission. In some embodiments, the subject has previously undergone, is undergoing, or will undergo a monotherapy course of treatment. In some embodiments, the subject has previously undergone, is undergoing, or will undergo radiation therapy. In some embodiments, the subject has previously undergone, is undergoing, or will undergo immunotherapy. In some embodiments, the subject has previously undergone, is undergoing, or will undergo chemotherapy. In some embodiments, the subject has previously undergone, is undergoing, or will undergo a platinum-based chemotherapy. In some embodiments, the subject has previously undergone, is undergoing, or will undergo a therapeutic regimen comprising administration of a KRAS modulator (e.g., KRAS inhibitor). In some embodiments, the subject has previously undergone, is undergoing, or will undergo a therapeutic regimen comprising administration of an anti-PD-1/PD-L1 inhibitor (e.g., checkpoint inhibitor).

The subject can have an advanced (e.g., primary or recurrent) solid tumor with a KRAS mutation (e.g., excluding KRAS Q61X) as assessed by molecular diagnostic using an appropriate clinically validated and/or FDA approved test and with no available standard of care or curative therapies. In some embodiments, the subject has a mutation in KRAS (excluding KRAS Q61X), as assessed by molecular diagnostic using an appropriate clinically validated and/or FDA approved test within at least one (1) year prior to the admission to the treatment as described herein.

In some embodiments, the subject has a mutation in KRAS, provided that the mutation is other than KRAS Q61X (e.g., the subject has a cancer characterized by a mutation in KRAS other than KRAS Q61X). In some embodiments, a KRAS protein includes a G12C, G12D, G12S, G12V, G12R, G12A, G13D, G13A, G13C, G13R, G13S, and/or G13V mutation (e.g., the subject has a cancer characterized by a G12C, G12D, G12S, G12V, G12R, G12A, G13D, G13A, G13C, G13R, G13S, and/or G13V mutation in KRAS). In some embodiments, subject has a KRAS mutation including a KRAS G12C mutation. In some embodiments, subject has a KRAS mutation including a KRAS G12A mutation, a KRAS G12D mutation, a KRAS G12F mutation, a KRAS G12I mutation, a KRAS G12L mutation, a KRAS G12R mutation, a KRAS G12S mutation, a KRAS G12V mutation, a KRAS G12Y mutation, a KRAS G13D mutation, or a combination thereof (e.g., the subject has a cancer characterized by a KRAS mutation including a KRAS G12C mutation, a KRAS G12A mutation, a KRAS G12D mutation, a KRAS G12F mutation, a KRAS G12I mutation, a KRAS G12L mutation, a KRAS G12R mutation, a KRAS G12S mutation, a KRAS G12V mutation, a KRAS G12Y mutation, a KRAS G13D mutation, or a combination thereof).

In some embodiments, the subject has the solid tumor progressed or recurred on or after at least one prior line of a systemic therapy including a platinum-based doublet chemotherapy and/or an anti-PD-1/PD-L1 therapy, each of which is given in monotherapy or both of which are given in combination therapy.

In some embodiments, the subject has the solid tumor progressed or recurred during a treatment of an anti-PD-1/PD-L1 therapy or within about 90 days after discontinuing an anti-PD-1/PD-L1 therapy.

In some embodiments, the subject has a measurable disease according to response evaluation criteria in solid tumors (RECIST).

In some embodiments, the subject has not previously participated in an interventional clinical study within a period of at least about four (4) weeks or five (5) half-lives of an agent used in the interventional clinical study, whichever is shorter prior to initiation of the treatment with the compound of formula (I) or (10b) in combination with nivolumab.

In some embodiments, the subject has not previously received a radiotherapy or a proton therapy including i) a limited field of radiation for palliation within a period of about one (1) week, or ii) a radiation to more than about 30% of bone marrow or a wide field of radiation within a period of about four (4) weeks, prior to initiation of the treatment with the compound of formula (I) or (10b) in combination with nivolumab.

In some embodiments, the subject has not taken or is not taking a) one or more of strong or moderate inducers or inhibitors of CYP3A4 and/or P-gp inducers or inhibitors (including herbal supplements or food products containing grapefruit juice, star fruit, or Seville oranges) (e.g., Appendix 3) within a period of about 14 days or five (5) half-lives, whichever is longer prior to initiation of the treatment with the compound of formula (I) or (10b) in combination with nivolumab; and/or b) a drug that is a known substrate of P-gp, breast cancer resistance protein (BCRP), multidrug and toxin extrusion protein (MATE)1, and/or MATE2-K transporters within a period of about 7 days prior to initiation of the treatment with the compound of formula (I) or (10b) in combination with nivolumab.

In some embodiments, the subject does not have inadequate organ functions including adequate hematological, renal, hepatic, and coagulating functions, as defined below:

### Hematological

a. White blood cell count <2,000/μL;
b. Absolute neutrophil count <1,500/μL;
c. Platelets <100,000/μL; and
d. Hemoglobin <9 g/dL without transfusion for ≤2 weeks or erythropoiesis-stimulating agents (e.g., Epo, Procrit) for ≤6 weeks.

### Renal

d. Serum creatinine >1.5×ULN, unless creatinine clearance ≥40 mL/min (measured or calculated using the Cockcroft-Gault formula)

### Hepatic

e. Serum total bilirubin ≥1.5×institutional upper limit of normal (ULN) or ≥3.0×institutional ULN if the patient has a diagnosis of Gilbert syndrome or hemolytic anemia as confirmed by the investigator; and
f. Aspartate aminotransferase/serum glutamic-oxaloacetic transaminase (AST/SGOT) and/or alanine aminotransferase/serum glutamic-pyruvic transaminase (ALT/SGPT) >2.5×ULN.

### Coagulation

g. International normalized ratio (INR) or prothrombin time (PT) >1.5×ULN unless the patient is receiving anticoagulant therapy and as long as PT or activated partial thromboplastin time (aPTT) is within the therapeutic range of intended use of anticoagulants; and
h. Activated partial thromboplastin time >1.5×ULN unless the patient is receiving anticoagulant therapy and as long as PT or aPTT is within the therapeutic range of intended use of anticoagulants.

In some embodiments, the subject does not have active hepatitis B infection, hepatitis C infection, or human immunodeficiency virus (HIV) infection with measurable viral load.

In some embodiments, the subject does not have has a life-threatening illness, medical condition, an active uncontrolled infection, or an organ system dysfunction (e.g., ascites, coagulopathy, or encephalopathy).

In some embodiments, the subject does not have one or more cardiac-related diseases or findings:
a) History of significant cardiovascular disease (e.g., cerebrovascular accident, myocardial infarction or unstable angina), within the last 6 months before starting the treatment;
b) Clinically significant cardiac disease, including New York Heart Association Class II or higher heart failure;
c) History of left ventricular ejection fraction (LVEF)<50% within the previous 12 months before starting the treatment;
d) Resting corrected QT interval (QTc) >470 msec, derived as the averaged from three electrocardiograms (ECGs), using the ECG machines provided; and/or
e) Any clinically significant abnormalities in rhythm, conduction, or morphology of resting ECG (e.g., third degree heart block, Mobitz Type II heart block, ventricular arrhythmias, uncontrolled atrial fibrillation).

In some embodiments, the subject has not been diagnosed of an additional invasive malignancy within the previous 3 years, provided that the additional invasive malignancy is other than curatively treated non-melanomatous skin cancer, superficial urothelial carcinoma, in situ cervical cancer, or any other curatively treated malignancy that is not expected to require treatment for recurrence during the course of the treatment with the compound of formula (I) or (10b) in combination with nivolumab.

In some embodiments, the subject does not have one or more untreated brain metastases from non-brain tumors.

In some embodiments, the subject who has had brain metastases resected or have received radiation therapy ending at least 4 weeks prior to the initiation of the treatment (e.g., Cycle 1, Day 1) with the compound of formula (I) or (10b) in combination with nivolumab is eligible, provided that the subject meets all of the following criteria prior to the initiation of the treatment: a) residual neurological symptoms related to the CNS treatment Grade ≤2; b) on a stable or decreasing dose of ≤10 mg daily prednisone (or equivalent) for at least 2 weeks prior to Cycle 1, Day 1, if applicable; and c) follow-up magnetic resonance imaging (MRI) within 4 weeks prior to Cycle 1, Day 1 shows no new lesions appearing.

In some embodiments, the subject has not undergone a major surgery within 4 weeks prior to the enrollment for the treatment with the compound of formula (I) or (10b) in combination with nivolumab, provided that the surgery or procedure is other than peripherally inserted central catheter line placement, thoracentesis, paracentesis, biopsies, or abscess drainage.

In some embodiments, the subject does not have a history of hypersensitivity to nivolumab or the compound of formula (I) or (10b), active or inactive excipients of nivolumab or the compound of formula (I) or (10b) or drugs with a similar chemical structure or class to either nivolumab or the compound of formula (I) or (10b), dependent on which combination the subject could receive.

In some embodiments, the subject does not have one or more additional activating mutations in BRAF V600X, PTPN11 (SHP2), and/or KRAS Q61X. In some embodiments, the subject does not have a tumor harboring one or more additional activating mutations in BRAF V600X, PTPN11 (SHP2), and/or KRAS Q61X.

In some embodiments, the subject is not previously treated with a PTPN11 inhibitor (e.g., SHP2 inhibitor), provided that the PTPN11 inhibitor is other than the compound of formula (I) or (10b). In some embodiments, the subject is not previously treated with a PTPN11 inhibitor selected from the group consisting of TNO-155, RMC-4630, RLY-1971, JAB-3068, JAB-3312, PF-07284892, and ERAS601. In some embodiments, the subject is not previously treated with the compound of formula (I) or (10b). In some embodiments, the subject has previously been treated with a SHP2 inhibitor including any one of TNO-155, RMC-4630, RLY-1971, JAB-3068, JAB-3312, PF-07284892, ERAS601, and the compound of formula (I) or (10b).

In some embodiments, the subject does not have a gastrointestinal illness (e.g., post gastrectomy, short bowel syndrome, uncontrolled Crohn's disease, celiac disease with villous atrophy, or chronic gastritis), which may preclude absorption of the compound of formula (I) or (10b).

In some embodiments, the subject is not on dialysis.

In some embodiments, the subject does not have a history of allogenic bone marrow transplant.

In some embodiments, the subject has not experienced a progressive disease (PD) within the first 120 days of initiating a treatment with an anti-PD-1/PD-L1 agent (e.g., primary refractory).

In some embodiments, the subject has not experienced ≥Grade 3 toxicity deemed related to prior anti-PD-1/PD-L1 treatment, which required discontinuation of therapy.

In some embodiments, the subject does not have a known or suspected autoimmune disease, provided that the autoimmune disease is other than type 1 diabetes, hypothyroidism only requiring hormone replacement, skin disorders (e.g., vitiligo, psoriasis, or alopecia) not requiring systemic treatment, or conditions not expected to recur in the absence of an external trigger.

In some embodiments, the subject does not have a condition requiring a systemic treatment with either corticosteroids (>10 mg prednisone equivalent) or other immunosuppressive medication within 14 days prior to the initiation of the treatment (e.g., Cycle 1, Day 1). In some embodiments, the subject is allowed to be on inhaled or topical steroids, and adrenal replacement steroids >10 mg prednisone equivalent, when the subject does not an active autoimmune disease.

In some embodiments, the subject has not received a live/attenuated vaccine within 30 days prior to the initiation of the treatment.

Further inclusion and exclusion criteria for subjects who may benefit from treatment with the compound of formula (I) or (10b) in combination with nivolumab, such as subjects enrolled in a Phase 1 Study of the SHP2 Inhibitor Compound (10b) in Combination with nivolumab, are described in Example 2.

In some embodiments, the subject meets all of inclusion criteria of 1) to 10) as described in Example 2. In some embodiments, the subject meets all of inclusion criteria of 1) to 10) as described in Example 2, provided that the subject does not meet any one of exclusion criteria of 1) to 22) as described in Example 2.

### III-4: Treatment Cycle and Dose Adjustment

Treatment with the compound (I) or (10b) in combination with nivolumab can include one or more treatment cycles (e.g., at least 1, 2, 3, or more treatment cycles). In some embodiments, the treatment includes one or more treatment cycles (e.g., at least 1, 2, 3, or more treatment cycles). In some embodiments, the treatment includes at least 2, 3, or more treatment cycles. In some embodiments, the treatment includes 2 to 3 treatment cycles. In some embodiments, the treatment includes 3 treatment cycles. In some embodiments, the treatment includes more than 3 treatment cycles.

In some embodiments, each of one or more treatment cycles has a duration of about 28 days; and the compound of formula (I) or (10b) is administered daily. In some embodiments, each of one or more treatment cycles has a duration of about 28 days; and nivolumab is administered every four (4) weeks. In some embodiments, each of one or more treatment cycles has a duration of about 28 days; the compound of formula (I) or (10b) is administered daily; and nivolumab is administered every four (4) weeks.

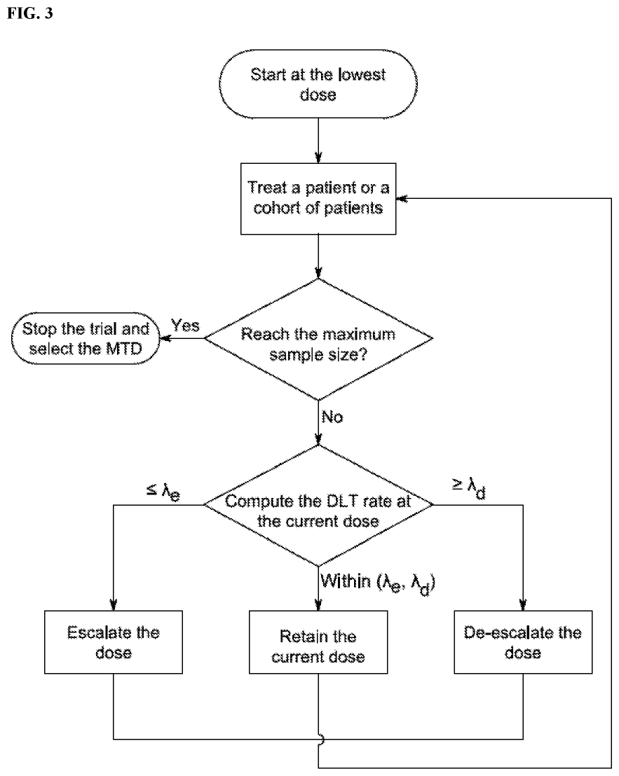

The treatment includes a dose escalation period, during which, after a previous treatment cycle, a dose of the compound of formula (I) or (10b) can be adjusted (e.g., dose escalation or de-escalation) or retained. Dose adjustment may be based at least in part on a safety evaluation (e.g., a dose-limiting toxicity (DLT) assessment). In some embodiments, the administration of the compound of formula (I) or (10b) includes one or more dose escalations, a dose retention, or a dose de-escalation, each of which is determined by a dose-limiting toxicity (DLT) assessment. In some embodiments, the administration of the compound of formula (I) or (10b) includes one or more dose escalations, a dose retention, or a dose de-escalation, each of which is determined by a dose-limiting toxicity (DLT) assessment, as described in Example 2, Example 5, and FIG. 3.

In some embodiments, the administration of the compound of formula (I) or (10b) includes a dose escalation after a previous treatment cycle, when a dose-limiting toxicity (DLT) rate is less than, e.g., about 19.7% determined by a DLT assessment. In some embodiments, the administration of the compound of formula (I) or (10b) includes a dose escalation in a second treatment cycle after a first treatment cycle, when a dose-limiting toxicity (DLT) rate is less than, e.g., about 19.7% determined by a DLT assessment. In some embodiments, the administration of the compound of formula (I) or (10b) includes a dose escalation in a third treatment cycle after a second treatment cycle, when a dose-limiting toxicity (DLT) rate is less than, e.g., about 19.7% determined by a DLT assessment.

In some embodiments, the administration of the compound of formula (I) or (10b) includes a dose de-escalation after a previous treatment cycle, when a dose-limiting toxicity rate is more than, e.g., about 29.8% determined by a DLT assessment. In some embodiments, the administration of the compound of formula (I) or (10b) includes a dose retention in a second treatment cycle after a first treatment cycle, when a dose-limiting toxicity rate is more than, e.g., about 29.8% determined by a DLT assessment. In some embodiments, the administration of the compound of formula (I) or (10b) includes a dose retention in a third treatment cycle after a second treatment cycle, when a dose-limiting toxicity rate is more than, e.g., about 29.8% determined by a DLT assessment.

In some embodiments, the administration of the compound of formula (I) or (10b) includes a dose retention after a previous treatment cycle, when a dose-limiting toxicity rate is in a range of from about 21.9% to about 29.8% determined by a DLT assessment. In some embodiments, the administration of the compound of formula (I) or (10b) includes a dose retention in a second treatment cycle after a first treatment cycle, when a dose-limiting toxicity rate is in a range of from about 21.9% to about 29.8% determined by a DLT assessment. In some embodiments, the administration of the compound of formula (I) or (10b) includes a dose retention in a third treatment cycle after a second treatment cycle, when a dose-limiting toxicity rate is in a range of from about 21.9% to about 29.8% determined by a DLT assessment.

After the dose escalation period, the treatment further includes a dose expansion/optimization period. In some embodiments of the dose expansion/optimization period, the compound of formula (I) or (10b) is administered at a dose regimen (e.g., Dose Regimen 1 or Dose Regimen 2) determined during the dose escalation period.

In some embodiments, during the dose expansion/optimization period, the administration of the compound of formula (I) or (10b) optionally includes one or more dose adjustments. In some embodiments, during the dose expansion/optimization period, the administration of the compound of formula (I) or (10b) optionally includes one or more dose adjustments; and the one or more dose adjustments are determined according to a safety evaluation by Safety Review Committee (SRC).

In any one of embodiments as described herein, nivolumab in a total dosage of about 480 mg every four (4) weeks is not adjusted (e.g., any dose escalation and/or de-escalation are not allowed during the treatment).

In some embodiments, dosing adjustments, delays, and discontinuations of the compound of formula (I) or (10b) and/or nivolumab are further based on the criteria of Example 3.

### III-5: Therapeutically Effective Amount/Administration

The compound of formula (I) or (10b) and nivolumab can be provided in jointly therapeutically effective amounts or in synergistically effective amounts, or each of which can be used at a dose less than when each is used alone. In some embodiments, the compound of formula (I) or (10b) and nivolumab are provided in jointly therapeutically effective amounts. In some embodiments, the compound of formula (I) or (10b) and nivolumab are provided in synergistically effective amounts. In some embodiments, the compound of formula (I) or (10b) and nivolumab are each used at a dose less than when each is used alone.

The compound of formula (I) or (10b) and nivolumab can be administered concomitantly or sequentially. In some embodiments, the compound of formula (I) or (10b) and nivolumab are administered concomitantly. In some embodiments, the compound of formula (I) or (10b) and nivolumab are administered sequentially. In some embodiments, the compound of formula (I) or (10b) is administered prior to the administration of nivolumab. In some embodiments, the compound of formula (I) or (10b) is administered after the administration of nivolumab.

In some embodiments, the therapeutically effective amount of the compound of formula (I) or (10b) is a total daily dosage of no more than about 2000 mg, on a salt-free and anhydrous basis. In some embodiments, the therapeutically effective amount is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (I) or (10b), on a salt-free and anhydrous basis, or any useful range therein. In some embodiments, the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis, or any useful range therein.

In some embodiments, the therapeutically effective amount of the compound of formula (10b) is a total daily dosage of no more than about 2000 mg, on a salt-free and anhydrous basis. In some embodiments, the therapeutically effective amount is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis, or any useful range therein. In some embodiments, the therapeutically effective amount is a total daily dosage of from about 250 mg to about 400 mg, from about 400 mg to about 550 mg, or from about 550 mg to about 700 mg of the compound of formula (10b), on a salt-free and anhydrous basis, or any useful range therein.

In some embodiments, the therapeutically effective amount of the compound of formula (10b) is a total daily dosage of about 250 mg, about 400 mg, or about 550 mg of the compound of formula (10b), on a salt-free and anhydrous basis. In some embodiments, the therapeutically effective amount is a total daily dosage of about 250 mg of the compound of formula (10b), on a salt-free and anhydrous basis. In some embodiments, the therapeutically effective amount is a total daily dosage of about 400 mg of the compound of formula (10b), on a salt-free and anhydrous basis. In some embodiments, the therapeutically effective amount is a total daily dosage of about 550 mg of the compound of formula (10b), on a salt-free and anhydrous basis.

In some embodiments, the therapeutically effective amount of the compound of formula (10b) is a total daily dosage of no more than about 2000 mg, on a salt-free and anhydrous basis; and the therapeutically effective amount of nivolumab is a total dosage of about 480 mg every four (4) weeks. In some embodiments, the therapeutically effective amount is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis, or any useful range therein; and the therapeutically effective amount of nivolumab is a total dosage of about 480 mg every four (4) weeks. In some embodiments, the therapeutically effective amount is a total daily dosage of from about 250 mg to about 400 mg, from about 400 mg to about 550 mg, or from about 550 mg to about 700 mg of the compound of formula (10b), on a salt-free and anhydrous basis, or any useful range therein; and the therapeutically effective amount of nivolumab is a total dosage of about 480 mg every four (4) weeks.

In some embodiments, the therapeutically effective amount of the compound of formula (10b) is a total daily dosage of about 250 mg, about 400 mg, or about 550 mg of the compound of formula (10b), on a salt-free and anhydrous basis; and the therapeutically effective amount of nivolumab is a total dosage of about 480 mg every four (4) weeks. In some embodiments, the therapeutically effective amount is a total daily dosage of about 250 mg of the compound of formula (10b), on a salt-free and anhydrous basis; and the therapeutically effective amount of nivolumab is a total dosage of about 480 mg every four (4) weeks. In some embodiments, the therapeutically effective amount is a total daily dosage of about 400 mg of the compound of formula (10b), on a salt-free and anhydrous basis; and the therapeutically effective amount of nivolumab is a total dosage of about 480 mg every four (4) weeks. In some embodiments, the therapeutically effective amount is a total daily dosage of about 550 mg of the compound of formula (10b), on a salt-free and anhydrous basis; and the therapeutically effective amount of nivolumab is a total dosage of about 480 mg every four (4) weeks.

In general, the compound of formula (I) or (10b) can be administered orally. In some embodiments, the compound of formula (10b) is administered orally. In some embodiments, the compound of formula (10b) in a tablet formulation is administered orally.

In general, nivolumab can be administered intravenously. In some embodiments, nivolumab is administered intravenously.

In general, the compound of formula (I) or (10b) can be administered once or multiple times (e.g., 2, 3, 4, or more times) daily. In some embodiments, the compound of formula (10b) is administered once, twice, three times, or four times daily. In some embodiments, the compound of formula (10b) is administered once daily. In some embodiments, the compound of formula (10b) is administered twice daily.

In general, nivolumab can be administered once or twice every four (4) weeks. In some embodiments, nivolumab is administered once every four (4) weeks.

In some embodiments, the compound of formula (I) or (10b) is administered orally; and nivolumab is administered intravenously. In some embodiments, the compound of formula (10b) is administered orally; and nivolumab is administered intravenously. In some embodiments, the compound of formula (10b) is administered once daily; and nivolumab is administered once every four (4) weeks.

The compound of formula (I) or (10b) can be in an oral dosage form in one or more dosage strengths, where the compound of formula (I) or (10b) is present in an amount of at least about 5 mg, 10 mg, 20 mg, 30 mg, 50 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (10b) is present in an amount of from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (10b) is present in an amount of about 5 mg, 10 mg, 30 mg, 50 mg, 100 mg, 150 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (10b) is present in an amount of about 30 mg, 50 mg, or 100 mg in each tablet, on a salt-free and anhydrous basis. In some embodiments of the tablet formulation, the compound of formula (10b) is present in an amount of about 30 mg in each tablet, on a salt-free and anhydrous basis. In some embodiments of the tablet formulation, the compound of formula (10b) is present in an amount of about 50 mg in each tablet, on a salt-free and anhydrous basis. In some embodiments of the tablet formulation, the compound of formula (10b) is present in an amount of about 100 mg in each tablet, on a salt-free and anhydrous basis.

In some embodiments, the compound of formula (10b) is administered once daily to provide a total daily dosage of no more than about 2000 mg of the compound of formula (10b). In some embodiments, the compound of formula (10b) is administered once daily to provide a total daily dosage of from about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis. In some embodiments, the compound of formula (10b) is administered once daily to provide a total daily dosage of from about 250 mg to about 400 mg, from about 400 mg to about 550 mg, or from about 550 mg to about 700 mg of the compound of formula (10b), on a salt-free and anhydrous basis. In some embodiments, the compound of formula (10b) is administered once daily to provide a total daily dosage of about 250 mg, about 400 mg, about 550 mg of the compound of formula (10b), on a salt-free and anhydrous basis.

In some embodiments, the compound of formula (10b) is administered once daily to provide a total daily dosage of no more than about 2000 mg of the compound of formula (10b); and nivolumab is administered once every four (4) weeks to provide a total dosage of about 480 mg. In some embodiments, the compound of formula (10b) is administered once daily to provide a total daily dosage of from about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis; and nivolumab is administered once every four (4) weeks to provide a total dosage of about 480 mg. In some embodiments, the compound of formula (10b) is administered once daily to provide a total daily dosage of from about 250 mg to about 400 mg, from about 400 mg to about 550 mg, or from about 550 mg to about 700 mg of the compound of formula (10b), on a salt-free and anhydrous basis; and nivolumab is administered once every four (4) weeks to provide a total dosage of about 480 mg. In some embodiments, the compound of formula (10b) is administered once daily to provide a total daily dosage of about 250 mg, about 400 mg, about 550 mg of the compound of formula (10b), on a salt-free and anhydrous basis; and nivolumab is administered once every four (4) weeks to provide a total dosage of about 480 mg.

In some embodiments, the compound of formula (10b) is administered once daily during each of one or more treatment cycles, as described herein. In some embodiments, nivolumab is administered once every four (4) weeks during each of one or more treatment cycles, as described herein. In some embodiments, the compound of formula (10b) is administered once daily and nivolumab is administered once every four (4) weeks during each of one or more treatment cycles, as described herein.

In general, the compound of formula (10b) is recommend to be administered to a subject without food (e.g., after an overnight fast (minimum 8 hours) followed by 2 hours of fasting after the dose is taken). The subject is allowed to have water except for one (1) hour before and after the administration and the subject is given with water (e.g., 240 mL) at the administration. In some embodiments, the compound of formula (10b) is administered to the subject without food, at least about 8 hours prior to the administration and at least about 2 hours post the administration.

In some embodiments, nivolumab is administered once every four (4) weeks intravenously, in about 30 minutes after administration of the compound of formula (10b).

### III-6: Efficacy

“A Phase 1 Study of the SHP2 Inhibitor Compound (10b) in Combination with Nivolumab in Patients with Advanced Non-Small Cell Lung Cancer with a KRAS Mutation” can evaluate the safety, tolerability, and efficacy of the compound of formula (10b) in combination with nivolumab to reduce or stabilize solid tumors in subjects, as summarized in Examples 2 and 6.

Administration of a therapeutically effective amount of the compound of formula (I) or (10b) in combination with a therapeutically effective amount of nivolumab can reduce or substantially eliminate cancers or solid tumors in subjects. In some embodiments, the therapeutically effective amount of formula (I) or (10b) in combination with nivolumab substantially eliminates the solid tumor. In some embodiments, the therapeutically effective amount of formula (I) or (10b) in combination with nivolumab reduces a volume of the solid tumor at least about 10%, about 20%, about 30%, about 40%, about 50%, about 60%, about 70%, about 80%, about 90%, or more. In some embodiments, the therapeutically effective amount of formula (I) or (10b) in combination with nivolumab reduces a volume of the solid tumor in a size of from about 10% to about 90%, from about 10% to about 80%, from about 10% to about 70%, from about 10% to about 60%, from about 10% to about 50%, from about 10% to about 40%, from about 10% to about 30%, from about 10% to about 20%, from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, from about 60% to about 70%, from about 70% to about 90%, from about 70% to about 80%, from about 80% to about 90%, or any range therein. In some embodiments, the therapeutically effective amount of formula (I) or (10b) in combination with nivolumab reduces a volume of the solid tumor about 10%, about 20%, about 30%, about 40%, about 50%, about 60%, about 70%, about 80%, or about 90%.

Administration of a therapeutically effective amount of the compound of formula (I) or (10b) in combination with a therapeutically effective amount of nivolumab can stabilize cancers or solid tumors in subjects. In some embodiments, the therapeutically effective amount of formula (I) or (10b) in combination with nivolumab stabilize the solid tumor.

Administration of a therapeutically effective amount of the compound of formula (I) or (10b) in combination with a therapeutically effective amount of nivolumab can maintain a reduction or stabilization of cancers or solid tumors in subjects for a period of time (e.g., 1 to 12 months). In some embodiments, the solid tumor is reduced or stabilized for a period of at least about one month with the therapeutically effective amount of the compound of formula (I) or (10b) in combination with nivolumab. In some embodiments, the solid tumor is reduced or stabilized for a period of at least about 2, 3, 4, 5, 6, 7, 8, 9, 10, 11, or 12 months with the therapeutically effective amount of the compound of formula (10b) in combination with nivolumab. In some embodiments, the solid tumor is reduced or stabilized for a period of from about 1 to about 12 months, from about 1 to about 6 months, from about 1 to about 3 months, or from about 1 to about 2 months.

In some embodiments, the subject is further evaluated to by one or more tests (e.g., tests according to Table 1, Table 5, Table 6, and Table 7) to provide overall assessments including plasma pharmacokinetic and/or pharmacodynamic profiles. Examples of such tests are described in, e.g., Examples 2 and 4.

In some embodiments, the subject is further evaluated for one or more biomarkers to determine a correlation of the one or more biomarkers to an antitumor response. Examples of such evaluation are described in Table 1 of Example 2 and Table 7 of Example 4.

### III-7: Combination Therapy for Treating NSCLC or CRC

In another aspect, the present disclosure provides a method of treating non-small cell lung cancer (NSCLC), the method including administering to the subject:
a) a therapeutically effective amount of a PTPN11 inhibitor, represented by formula (I):

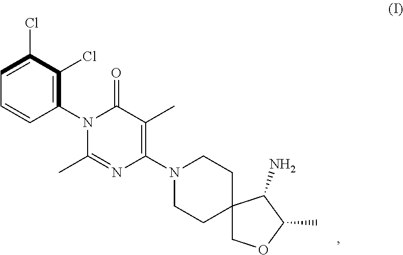

or a pharmaceutically acceptable salt, hydrate, solvate, stereoisomer, conformational isomer, or a combination thereof;
b) a therapeutically effective amount of nivolumab.

In another aspect, the present disclosure provides a method of treating colorectal cancer (CRC), the method including administering to the subject:
a) a therapeutically effective amount of a PTPN11 inhibitor, represented by formula (I):

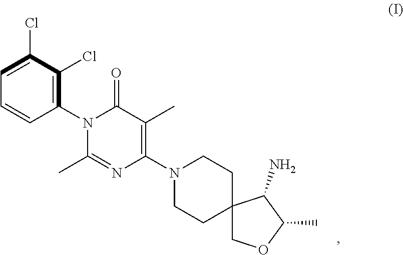

or a pharmaceutically acceptable salt, hydrate, solvate, stereoisomer, conformational isomer, or a combination thereof;
b) a therapeutically effective amount of nivolumab.

The compound of formula (I) is described according to Section III-1: Compound of Formula (I). In some embodiments, the compound of formula (I) is any of embodiments as described in Section III-1: Compound of Formula (I). In some embodiments, the PTPN11 inhibitor of formula (I) is the compound of formula (10b).

The non-small cell lung cancer (NSCLC) or colorectal cancer (CRC) is described according to Section III-2: Cancer/Solid Tumor. In some embodiments, the NSCLC or CRC is any of embodiments as described in Section III-2: Cancer/Solid Tumor, wherein the cancer/solid tumor is NSCLC or CRC.

The subject is described according to Section III-3: Subject. In some embodiments, the subject is any of embodiments as described in Section III-3: Subject. In some embodiments, the subject is any of embodiments as described in Section III-3: Subject, wherein the subject has NSCLC or CRC.

The treatment cycle and dose adjustment are described according to Section III-4: Treatment Cycle and Dose Adjustment. In some embodiments, the therapeutically effective amount and/or administration are any one of embodiments as described in Section III-4: Treatment Cycle and Dose Adjustment.

The therapeutically effective amount and/or administration are described according to Section III-5: Therapeutically Effective Amount/Administration. In some embodiments, the therapeutically effective amount and/or administration are any one of embodiments as described in Section III-5: Therapeutically Effective Amount/Administration.

The efficacy of the treatment is described according to Section III-6: Efficacy. In some embodiments, the efficacy of the treatment is any one of embodiments as described in Section III-6: Efficacy, wherein the cancer/solid tumor is NSCLC or CRC.

### IV. Composition

The oral dosage form including the compound of formula (I) or (10b) can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the patient.

For preparing oral dosage forms including the compound of formula (I) or (10b), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% to 70% of the compound of formula (I) or (10b), or from about 10% to about 70% of the compound of formula (I) or (10b). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the compound of formula (I) or (10b) mixed with a filler or binders such as lactose or starches, lubricants such as talc or magnesium stearate, and, optionally, stabilizers. In soft capsules, the compound of formula (I) or (10b) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of formula (I) or (10b) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the compound of formula (I) or (10b) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula (I) or (10b) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J Pharmacol. Exp. Ther. 281:93-102, 1997. The pharmaceutical formulations including the compound of formula (I) or (10b) can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

### V. Kits

In another aspect, the present disclosure provides a kit for treating cancer in a subject, the kit including:
a) a therapeutically effective amount of a compound represented by formula (I); and
b) a therapeutically effective amount of nivolumab, together with instruction for effective administration,
wherein the compound of formula (I) is as defined and described herein.

The cancer and/or solid tumor are described according to Section III-2: Cancer/Solid Tumor. In some embodiments, the cancer and/or solid tumor are any of embodiments as described in Section III-2: Cancer/Solid Tumor.

The subject is described according to Section III-3: Subject. In some embodiments, the subject is any of embodiments as described in Section III-3: Subject.

The compound of formula (I) is described according to Section III-1: Compound of Formula (I). In some embodiments, the compound of formula (I) is any of embodiments as described in Section III-1: Compound of Formula (I). In some embodiments, the PTPN11 inhibitor of formula (I) is the compound of formula (10b).

In some embodiments, the kit includes instructions for administration of the compound of formula (I) or (10b) and nivolumab. In some embodiments, the kit includes instructions for administration of the compound of formula (10b) and nivolumab. In some embodiments, such instructions include directions relating to safety provisions as well as timing and amounts of administration of the compound of formula (I) or (10b) and nivolumab. In some embodiments, such instructions include directions relating to safety provisions as well as timing and amounts of administration of the compound of formula (10b) and nivolumab.

### Vi. List of Abbreviations

| Abbreviation | Definition |
|---|---|
| (e)CRF | (electronic) case report form |
| (p)ERK | (phosphorylated) extracellular signal-regulated kinase |
| ADA | anti-drug antibody |
| AE | adverse event |
| ALK | anaplastic lymphoma kinase |
| ALT | alanine aminotransferase (or transaminase) |
| ANC | absolute neutrophil count |
| aPTT | activated partial thromboplastin time |
| AST | aspartate aminotransferase (or transaminase) |
| AUC | area under the concentration-time curve |
| BBP | BridgeBio Pharma |
| BCRP | breast cancer resistance protein |
| BICR | blinded independent central review |
| BMS | Bristol Myers Squibb |
| BOIN | Bayesian optimal interval |
| CI | confidence interval |
| Cₘₐₓ | maximum drug concentration |
| CNS | central nervous system |
| CR | complete response(s) |
| CRO | contract research organization |
| CT | computed tomography |
| CTCAE | Common Terminology Criteria for |
| | Adverse Events |
| CYP | cytochrome P450 |
| DLT | dose-limiting toxicity |
| DNA | deoxyribonucleic acid |
| DOR | duration of response |
| DUSP6 | dual specificity phosphatase 6 |
| ECG | electrocardiogram |
| ECHO | echocardiogram |
| ECOG | Eastern Cooperative Oncology Group |
| EDC | electronic data capture (system) |
| EGFR | epidermal growth factor receptor |
| EORTC | European Organization for Research |
| | and Treatment of Cancer |
| EOS | end of study |
| EOT | end of treatment |
| FDA | Food and Drug Administration |
| FSH | follicle-stimulating hormone |
| GCP | Good Clinical Practice |
| GLP | Good Laboratory Practice |
| GMP | Good Manufacturing Practice |
| HBV | hepatitis B virus |
| HCV | hepatitis C virus |
| HIV | human immunodeficiency virus |
| IACS | Institute for Applied Cancer Science |
| IB | Investigator's Brochure |
| ICF | informed consent form |
| ICH | International Conference on Harmonization |
| IEC | Independent Ethics Committee |
| INR | international normalized ratio |
| IRB | Institutional Review Board |
| IRR | infusion-related reaction |
| IV | intravenous |
| KRAS | Kirsten rat sarcoma viral oncogene homolog |
| LD (or LDi) | longest diameter |
| LVEF | left ventricular ejection fraction |
| MAPK | mitogen-activated protein kinase |
| MATE | multidrug and toxin extrusion protein |
| MRI | magnetic resonance imaging |
| MTD | maximum tolerated dose |
| MUGA | multiple-gated acquisition scan (radionuclide angiography) |
| NCI | National Cancer Institute |
| NGS | next generation sequencing |
| NSCLC | non-small cell lung cancer |
| OATP | organic anion transporter polypeptide |
| ORR | objective response rate |
| OS | overall survival |
| PD | progressive disease |
| PD-1 | programmed cell death-1 |
| PD-L1 | programmed cell death ligand-1 |
| PET | positron emission tomography |
| PFS | progression free survival |
| P-gp | P-glycoprotein |
| PI | package insert |
| PK | pharmacokinetic |
| PR | partial response |
| PS | performance status |
| PT | prothrombin time |
| PTPN11 | protein tyrosine phosphatase non-receptor |
| | type 11, the gene encoding SHP2 |
| QD | once daily |
| RAF | rapidly accelerated fibrosarcoma |
| RAS | any of a family of genes that undergo |
| | mutation to oncogenes and especially to |
| | some commonly linked to human cancers |
| | (as of the colon, lung, and pancreas) |
| REB | Research Ethics Board |
| RECIST | Response Evaluation Criteria in Solid Tumors |
| RNA | ribonucleic acid |
| RP2D | recommended phase 2 dose |
| RTK | receptor tyrosine kinase |
| SAE | serious adverse event |
| SAP | Statistical Analysis Plan |
| SD | stable disease |
| SGOT | serum glutamic-oxaloacetic transaminase |
| SGPT | serum glutamic-pyruvic transaminase |
| SHP2 | Src homology-2-containing protein tyrosine phosphatase 2 |
| SRC | Safety Review Committee |
| STD10 | severely toxic dose in 10% of animals |
| SUSAR | suspected unexpected serious adverse reaction |
| TEAE | treatment-emergent adverse event |
| Tₘₐₓ | time to reach maximum drug concentration |
| | following drug administration |
| ULN | upper limit of normal |
| US | United States |

### VII. Examples

### Example 1: Evaluation of Combination of PD-1/PD-L1 Inhibitor and PTPN11 Inhibitor

### CD8+ Activation in Peripheral Blood Mononuclear Cells (PBMCs)

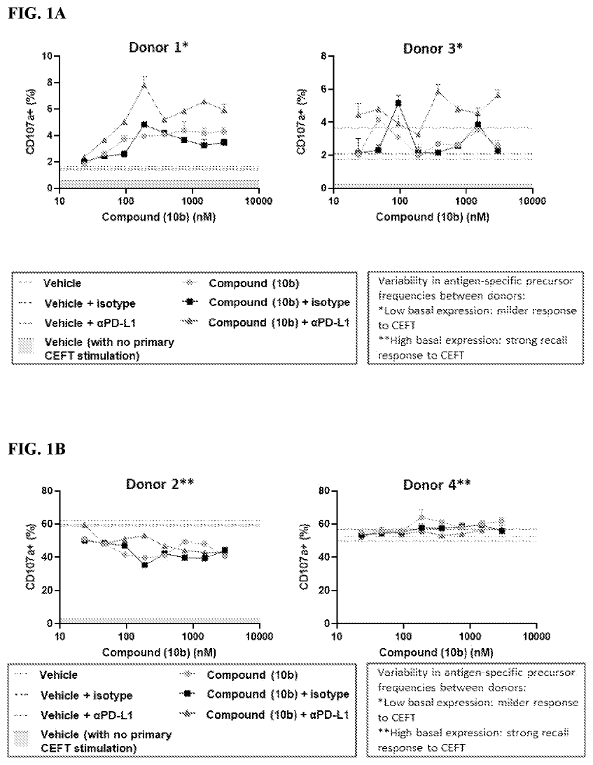

A study was performed to evaluate the effect of the SHP2 inhibitor Compound (10b) alone and in combination with αPD-L1 on T cell cytokine production and killing potential in response to antigenic stimulation. PBMCs from four healthy donors were stimulated with a CEFT peptide pool and treated with vehicle, isotype control, αPD-L1, a titration of Compound (10b), or a titration of Compound (10b) with the addition of either isotype control or αPD-L1. After the initial CEFT stimulation, the cultures were rested and cytokines were added to expand the number of responding CD8+ T cells. Cells were then restimulated with the CEFT peptide pool for 24 hours, and the frequency of CD107a+ cells as a percentage of total CD8+ T cells was assessed by flow cytometry. In addition, at 5 days post stimulation, the supernatents were harvested and the concentration of IFNγ was measured by TR-FRET. FIG. 1A-FIG. 1B show results from this study. The results showed that donors 2 and 4 had strong recall responses to CEFT stimulation, while donors 1 and 3 had milder responses. The addition of the SHP2 inhibitor Compound (10b) to the assay resulted in mixed effects between donors, which was likely to be caused by the variability in antigen-specific precursor frequencies between donors.

For donor 1, there was a clear Compound (10b) dose-dependent effect and the combination of Compound (10b) and αPD-L1 showed synergy, resulting in improved activation of CD8+ T cells, as indicated by higher levels of CD107a+CD8+ T cells. For donor 3, there was some Compound (10b) effect and the combination of Compound (10b) and αPD-L1 showed synergy, resulting in improved activation of CD8+ T cells, as indicated by higher levels of CD107a+CD8+ T cells. For donor 2, the addition of Compound (10b) resulted in a dose dependent decrease in the percentage of CD107a+CD8+ T cells, and there was no synergy with the combination of Compound (10b) and αPD-L1. For donor 4, there was no Compound (10b) effect or synergy with the combination of Compound (10b) and αPD-L1, as indicated by no change in CD107a+CD8+ T cell levels. Finally, the IFNγ levels 5 days after CEFT stimulation were inconsistent across donors (data not shown). These results indicate a possible role for Compound (10b) and the combination of Compound (10b) and αPD-L1I in specifically enhancing weak or sub-optimal CD8+ T cell responses.

### Example 2: A Phase 1 Study of the SHP2 Inhibitor Compound (10b) in Combination with the Programmed Death Receptor-1 Blocking Antibody Nivolumab in Patients with Advanced Non-Small Cell Lung Cancer with a KRAS Mutation—Study Synopsis

### Study Synopsis

Study Title: A Phase 1 Study of the SHP2 Inhibitor Compound (10b) in combination with the
Programmed Death Receptor-1 Blocking Antibody Nivolumab in Patients with Advanced Non-
Small Cell Lung Cancer with a KRAS Mutation
Study Phase: Phase 1a Dose Escalation, Phase 1b Dose Expansion
OBJECTIVES
Phase 1a Dose Escalation
Primary Objective: The primary objective for Phase 1a Dose Escalation is to evaluate the safety,
tolerability, and recommended phase 2 dose (RP2D) of Compound (10b), a SHP2 inhibitor,
when used in combination with nivolumab in patients with advanced non-small cell lung cancer
(NSCLC) with a KRAS mutation who have failed standard of care treatment.
Secondary Objectives: The secondary objectives for Phase 1a Dose Escalation are to:

Assess preliminary antitumor activity of Compound (10b) in combination with
nivolumab (as defined by objective response rate [ORR, complete response (CR) +
partial response (PR) rate], duration of response [DOR], and progression free survival
[PFS] according to Response Evaluation Criteria in Solid Tumors (RECIST) v1.1, and as
assessed by investigator, and overall survival [OS])
Characterize the pharmacokinetics (PK) of Compound (10b) and nivolumab given in
combination
Characterize circulating and intratumoral target engagement (pharmacodynamic activity)
of Compound (10b) in combination with nivolumab
Characterize the immunogenicity of nivolumab when given in combination with
Compound (10b)

Exploratory Objective: The exploratory objective for Phase 1a Dose Escalation is to assess
peripheral and intratumoral biomarkers that correlate with response.
Phase 1b Dose Expansion
Primary Objective: The primary objective for Phase 1b Dose Expansion is to evaluate the

antitumor activity of Compound (10b), as defined by the ORR (per investigator)

according to RECIST v1.1, when used in combination with nivolumab in patients with advanced
NSCLC with a KRAS mutation who have failed standard of care treatment.
Secondary Objectives: The secondary objectives for Phase 1b Dose Expansion are to:

Assess additional measures of antitumor activity of Compound (10b) in combination with
nivolumab, including ORR (per blinded independent central review [BICR]) and DOR
and PFS (per investigator and BICR), as defined by RECIST v1.1, and OS
Assess safety and tolerability of Compound (10b) at the RP2D, in combination with
nivolumab
Characterize the PK of Compound (10b) and nivolumab given in combination
Characterize circulating and intratumoral target engagement (pharmacodynamic activity)
of Compound (10b) in combination with nivolumab
Characterize the immunogenicity of nivolumab when given in combination with
Compound (10b)

Exploratory Objective: The exploratory objective for Phase 1b Dose Expansion is to assess
peripheral and intratumoral biomarkers that correlate with response.
Study Centers (US Sites Only):
Phase 1a Dose Escalation: Approximately 6
Phase 1b Dose Expansion: Approximately 20
Number of Patients Planned:
Overall, approximately 45 patients are planned for study enrollment: approximately 20 patients
in Phase 1a Dose Escalation and approximately 25 patients in Phase 1b Dose Expansion.
However, during Phase 1a Dose Escalation, additional patients in existing cohorts or an
additional cohort of 5-7 patients may be enrolled if deemed necessary by the SRC to evaluate the
safety, tolerability, and the RP2D. During Phase 1b Dose Expansion, an additional cohort of 25
patients may be enrolled if deemed necessary by the SRC to optimize the selection of the RP2D.
PATIENT ELIGIBILITY
INCLUSION CRITERIA
Patients must meet all of the inclusion criteria listed below (as applicable) to be eligible for
participation in the study.

| | |
|---|---|
| 1. | Individuals ≥18 years old and be willing and able to provide signed informed consent at |
| | the Screening visit as well as comply with all study visits and requirements through the |
| | end of the study. |
| 2. | Have documentation of a KRAS mutation from local or central laboratory testing in |
| | tumor samples collected within the 1 year prior to screening. |
| 3. | Have measurable disease by RECIST v1.1. |
| 4. | Have minimum life expectancy of >12 weeks. |
| 5. | Women must have a negative serum human chorionic gonadotropin test if of childbearing |
| | potential or have had prior tubal ligation (≥1 year prior to screening), a total |
| | hysterectomy, or have had menopause (defined as 12 consecutive months of amenorrhea |
| | and confirmed by follow up hormone level assessment). |
| 6. | Patients of childbearing potential must use 2 methods of contraception for the duration of |
| | the study and for at least 5 months after the last dose of study treatment for female |
| | patients or 105 days after the last dose of study treatment for male patients, whichever is |
| | later for the individual patient. Female patients should not become pregnant or breastfeed |
| | while on this study. Female and male patients must also agree not to donate eggs (ova, |
| | oocytes) or sperm for the purpose of reproduction for at least 5 months or 105 days after |
| | the last dose of study treatment, respectively. Acceptable methods of contraception are |
| | fully described in Appendix 2. |
| 7. | Have histologically documented, locally advanced and unresectable, or metastatic |
| | NSCLC. |
| 8. | Have progression or disease recurrence on or after at least one prior line of systemic |
| | therapy, which must include platinum-based doublet chemotherapy and anti-PD-(L)1 |
| | therapy, either given as one line or individual lines of therapy. |
| 9. | Experienced radiologically documented progressive or recurrent disease occurring either |
| | during treatment or within 90 days after discontinuing anti-PD-(L)1 therapy. |
| 10. | Patients must have an Eastern Cooperative Oncology Group (ECOG) performance status |
| | (PS) 0-1 (see Appendix 1) with no deterioration over the previous 2 weeks. |

EXCLUSION CRITERIA
Patients who meet any of the exclusion criteria listed below will not be eligible for participation
in the study.

| 1. | Have participated in an interventional clinical study within the last 4 weeks OR, if | | |
|---|---|---|---|
| | applicable, be within 5 times the half-life of the investigational study drug, whichever is | | |
| | lesser, prior to the C1D1 visit. Patients should always adhere to other eligibility criteria | | |
| | that apply to specified concomitant medication regarding washout periods as specified | | |
| | below. | | |
| 2. | Have received radiotherapy or proton therapy with a limited field of radiation for | | |
| | palliation within 1 week of the start of study treatment, OR radiation to more than 30% of | | |
| | the bone marrow or with a wide field of radiation within 4 weeks of the start of study | | |
| | treatment. | | |
| 3. | Have taken any of the following (see Appendix 3 for further details): | | |
| | a. | Strong or moderate inducers or inhibitors of cytochrome P450 (CYP) 3A4 or P- | |
| | | glycoprotein (P-gp) inducers or inhibitors (including herbal supplements or food | |
| | | products containing grapefruit juice, star fruit, or Seville oranges) within 14 days or 5 | |
| | | half-lives (whichever is longer) of Cycle 1 Day 1, and/or | |
| | b. | Drugs that are known substrates of P-gp, breast cancer resistance protein (BCRP), | |
| | | multidrug and toxin extrusion protein (MATE) 1 or MATE2-K, unless stopped 7 days | |
| | | prior to Cycle 1 Day 1 and for the study duration. | |
| 4. | Have inadequate organ function as defined below: | | |
| | Hematological | | |
| | | e. | White blood cell count <2,000/μL |
| | | f. | Absolute neutrophil count <1,500/μL |
| | | g. | Platelets <100,000/μL |
| | | h. | Hemoglobin <9 g/dL without transfusion for ≤2 weeks or erythropoiesis- |
| | | | stimulating agents (e.g., Epo, Procrit) for ≤6 weeks |
| | Renal | | |
| | | e. | Serum creatinine >1.5 × ULN, unless creatinine clearance ≥ 40 mL/min |
| | | | (measured or calculated using the Cockcroft-Gault formula) |
| | Hepatic | | |
| | | g. | Serum total bilirubin ≥1.5 × institutional upper limit of normal (ULN) or ≥3.0 × |
| | | | institutional ULN if the patient has a diagnosis of Gilbert syndrome or hemolytic |
| | | | anemia as confirmed by the investigator |
| | | h. | Aspartate aminotransferase/serum glutamic-oxaloacetic transaminase |
| | | | (AST/SGOT) and/or alanine aminotransferase/serum glutamic-pyruvic |
| | | | transaminase (ALT/SGPT) >2.5 × ULN |
| | Coagulation | | |
| | | i. | International normalized ratio (INR) or prothrombin time (PT) >1.5 × ULN unless |
| | | | the patient is receiving anticoagulant therapy and as long as PT or activated partial |
| | | | thromboplastin time (aPTT) is within the therapeutic range of intended use of |
| | | | anticoagulants |
| | | j. | Activated partial thromboplastin time >1.5 × ULN unless the patient is receiving |
| | | | anticoagulant therapy and as long as PT or aPTT is within the therapeutic range of |
| | | | intended use of anticoagulants |

| 5. | Have active Hepatitis B infection (defined by the presence of hepatitis B surface antigen | |
|---|---|---|
| | [HBsAg] or the presence of hepatitis B virus [HBV] DNA), hepatitis C infection (defined | |
| | by the presence of hepatitis C virus [HCV] antibody and positive HCV RNA), or human | |
| | immunodeficiency virus (HIV) infection with measurable viral load. | |
| 6. | Have a life-threatening illness, medical condition, active uncontrolled infection, or organ | |
| | system dysfunction (such as ascites, coagulopathy, or encephalopathy), or other reasons | |
| | which, in the investigator's opinion, could compromise the participating patient's safety, | |
| | or interfere with or compromise the integrity of the study outcomes. | |
| 7. | Have any of the following cardiac-related issues or findings: | |
| | a. | History of significant cardiovascular disease, such as cerebrovascular accident, |
| | | myocardial infarction or unstable angina, within the last 6 months before starting |
| | | study treatment. |
| | b. | Clinically significant cardiac disease, including New York Heart Association Class II |
| | | or higher heart failure. |
| | c. | History of left ventricular ejection fraction (LVEF) <50% within the previous 12 |
| | | months before starting study treatment. |
| | d. | Resting corrected QT interval (QTc) >470 msec, derived as the averaged from three |
| | | electrocardiograms (ECGs), using the ECG machines provided by the sponsor for |
| | | study purposes. |
| | e. | Any clinically significant abnormalities in rhythm, conduction, or morphology of |
| | | resting ECG (e.g., third degree heart block, Mobitz Type II heart block, ventricular |
| | | arrhythmias, uncontrolled atrial fibrillation). |

| | |
|---|---|
| 8. | Have a diagnosis of another invasive malignancy within the previous 3 years other than |
| | curatively treated non-melanomatous skin cancer, superficial urothelial carcinoma, in situ |
| | cervical cancer, or any other curatively treated malignancy that is not expected to require |
| | treatment for recurrence during the course of the study. |
| 9. | Have untreated brain metastases from non-brain tumors. Patients who have had brain |
| | metastases resected or have received radiation therapy ending at least 4 weeks prior to |
| | Cycle 1, Day 1 are eligible if they meet all of the following criteria prior to first dose of |
| | study medication: a) residual neurological symptoms related to the CNS treatment Grade |
| | ≤2; b) on a stable or decreasing dose of ≤10 mg daily prednisone (or equivalent) for at |
| | least 2 weeks prior to Cycle 1, Day 1, if applicable; and c) follow-up magnetic resonance |
| | imaging (MRI) within 4 weeks prior to C1D1 shows no new lesions appearing. |
| 10. | Have undergone major surgery within 4 weeks prior to study enrollment. |
| | Note: This does not include patients who have had procedures such as peripherally |

inserted central catheter line placement, thoracentesis, paracentesis, biopsies, or
abscess drainage.

| | |
|---|---|
| 11. | Have a history of hypersensitivity to nivolumab or Compound (10b), active or inactive |
| | excipients of nivolumab or Compound (10b) or drugs with a similar chemical structure or |
| | class to either nivolumab or Compound (10b), dependent on which combination the |
| | patient could receive. |
| 12. | Have tumors harboring known activating mutations in BRAF V600X, PTPN11 (SHP2), |
| | or KRAS Q61X. |
| 13. | Have previously received a SHP2 inhibitor (eg, TNO-155, RMC-4630, RLY-1971, JAB- |
| | 3068, JAB-3312 and PF-07284892). |
| 14. | Have gastrointestinal illness that, in the opinion of the investigator, would preclude |
| | absorption of Compound (10b) (e.g., post gastrectomy, short bowel syndrome, |
| | uncontrolled Crohn's disease, celiac disease with villous atrophy, or chronic gastritis). |
| 15. | Are on dialysis. |
| 16. | Have a history of allogenic bone marrow transplant. |
| 17. | Are unable to swallow oral medications (capsules, tablets) without chewing, breaking, |
| | crushing, opening, or otherwise altering the product dosage form. |
| 18. | Experienced progressive disease (PD) within the first 120 days of initiating treatment |
| | with an anti-PD-(L)1 agent (e.g., primary refractory). |
| 19. | Experienced ≥Grade 3 toxicity deemed related to prior anti-PD-(L)1 treatment which |
| | required discontinuation of therapy. |
| 20. | Have known or suspected autoimmune disease, except patients are permitted to enroll in |
| | cases of type 1 diabetes, hypothyroidism only requiring hormone replacement, skin |
| | disorders (e.g., vitiligo, psoriasis, or alopecia) not requiring systemic treatment, or |
| | conditions no expected to recur in the absence of an external trigger. |
| 21. | Have a condition requiring systemic treatment with either corticosteroids (>10 mg |
| | prednisone equivalent) or other immunosuppressive medication within 14 days of Cycle |
| | 1 Day 1. Inhaled or topical steroids, and adrenal replacement steroids >10 mg prednisone |
| | equivalent are permitted in the absence of active autoimmune disease. |
| 22. | Have received any live/attenuated vaccine within 30 days of first study treatment. |

STUDY DESIGN
Overview: This is a Phase 1 study of Compound (10b), a SHP2 inhibitor, in combination with
nivolumab, a PD-1 antibody, in patients with NSCLC and a KRAS mutation. The study involves
2 parts: Phase 1a Dose Escalation and Phase 1b Dose Expansion. Patients in both parts will have
advanced NSCLC with a KRAS mutation and had at least one previous line of treatment which
included platinum-based doublet chemotherapy and anti-PD-(L)1 therapy, either given as one
line or individual lines of therapy.
Enrollment will begin with Phase 1a Dose Escalation; enrollment will continue into Phase 1b
Dose Expansion after completion of Phase 1a Dose Escalation. All patients will be enrolled using
interactive response technology.
Each part of the study consists of a 30-day screening period; a treatment period consisting of
sequential consecutive treatment cycles; post-treatment follow-up visits at 28 days and 100 days
following the patient's last dose of study treatment; and a long-term follow up period. In both
parts, dosing will continue for 2 years unless the patient is discontinued from study treatment or
is withdrawn from the study. End of Treatment (EOT) Follow-up Visits will be conducted 28
days and 100 days following the patient's last dose of study treatment, unless one of the
following occurs: death, loss to follow-up, or withdrawal of consent. Long-term follow-up (with
telephone contact every 3 months via telephone) will continue for up to 3 years unless the patient
is withdrawn from the study. Additional long-term follow-up information is provided in Section
4.1.5 of the clinical protocol).
Phase 1a Dose Escalation
During Phase 1a Dose Escalation, dose escalation will follow a Bayesian optimal interval
(BOIN) design. Three dose levels of Compound (10b) are planned in Phase 1a Dose Escalation:
250 mg, 400 mg, and 550 mg. Nivolumab will be administered in combination with Compound
(10b) at the Food and Drug Administration approved dose for nivolumab monotherapy (480 mg
once every 4 weeks). Approximately 20 patients are anticipated for Phase 1a Dose Escalation.

| Dose Level/Number | | |
|---|---|---|
| of Patients ^{a} | Compound (10b) ^{b} | Nivolumab |
| Level 1 (n = 5-7) | 250 mg QD | 480 mg every 4 week |
| Level 2 (n = 5-7) | 400 mg QD | 480 mg every 4 week |
| Level 3 (n = 5-7) | 550 mg QD | 480 mg every 4 week |

^{a} Additional patients may be enrolled in existing cohorts if deemed necessary by the SRC to
evaluate the safety, tolerability, and the RP2D.
^{b} Dose escalation, an additional dose level, alternative dose levels, and/or alternative dose
schedules may be enrolled following SRC review of the totality of the data and after the SRC
approves or makes a recommendation regarding the next dose cohort.
Abbreviations: QD = once daily; RP2D = recommended phase 2 dose; SRC = Safety Review
Committee; wk = week
For each dose level of Phase 1a Dose Escalation, an SRC will evaluate safety (including dose-
limiting toxicities [DLTs]) and available PK and pharmacodynamic data at the end of the DLT
Assessment Period. The SRC will make dosing and escalation decisions in accordance with the
escalation and de-escalation rules of the BOIN design using a target toxicity rate of 25% with
escalation and de-escalation thresholds of 19.7% and 29.8%, respectively. An additional dose
level, alternative dose levels and/or alternative dose schedules may be enrolled following SRC
review of the totality of the data and after the SRC approves or makes a recommendation
regarding the next dose cohort. Further, at any point additional patients may be enrolled in
existing cohorts if deemed necessary by the SRC to evaluate the safety, tolerability, and the
RP2D.
Phase 1b Dose Expansion
In Phase 1b Dose Expansion, patients will receive Compound (10b) at the RP2D from Phase 1a
Dose Escalation, in combination with nivolumab, to assess preliminary antitumor activity as
defined by ORR per RECIST v1.1. Based on an SRC decision following review of the safety and
PK/PD data from Dose Escalation, a second cohort at another dose of Compound (10b) assessed
in escalation may be enrolled to optimize dose selection. Furthermore, the dose during Dose
Expansion may be adjusted based on an SRC decision following review of the totality of the
data.
With a sample size of 25 patients, an exact binomial test with a nominal 5% one-sided
significance level will have 80.65% power to detect the difference between the null hypothesis
response rate of 10% and the alternative response rate of 30%.
Protocol Amendment
Pending emerging data from this study and others, the sponsor may amend this protocol to
expand the population or include another combination, including the potential for a Compound
(10b) + nivolumab + KRAS^{G12C} i triplet. The decision and timing to amend the protocol will be
made after sufficient safety and PK/PD data are available from the doublet combination. Proper
justification and details of the potential amendment will be submitted and only upon approval
will the amendment be initiated.
Test Product, Dose, and Administration: In each 28-day treatment cycle, Compound (10b)
will be taken QD, in combination with intravenous (IV) nivolumab every 28 days. Absent a
reason to interrupt dosing, a new cycle will be initiated immediately upon completion of the prior
cycle.
Compound (10b) will be administered as oral capsules of 50 and 100 mg. Nivolumab (480 mg
every 4 weeks) will be administered by IV infusion approximately 30 minutes after
administration of Compound (10b).
Reference Therapy, Dose, and Administration: Not applicable
CRITERIA FOR EVALUATION
Phase 1a Dose Escalation
The primary endpoint for Phase 1a Dose Escalation is incidence and severity of treatment-
emergent adverse events (TEAEs), serious adverse events (SAEs), and DLTs.
The secondary endpoints are:

Antitumor activity, as defined by ORR (CR + PR), DOR, and PFS, per RECIST v1.1 and
as assessed by investigator, and OS,
PK parameters (including, but not limited to area under the curve [AUC], maximum drug
concentration [Cₘₐₓ], time to Cₘₐₓ [Tₘₐₓ], half-life) of Compound (10b) and nivolumab
from plasma or serum concentration-time data,
Pharmacodynamic parameters (including but not limited to raw, normalized, and/or
baseline adjusted analyte signal in circulating and intratumoral target engagement
biomarkers of Compound (10b) activity in combination with nivolumab)
Measurement of anti-drug antibodies (ADA) to nivolumab

Biomarkers that correlate with response to treatment will be an exploratory endpoint.
Phase 1b Dose Expansion
The primary endpoint for Phase 1b Dose Expansion is ORR (per investigator), as defined by
RECIST v1.1.
The secondary endpoints are:

DOR (per investigator) as defined by RECIST v1.1,
PFS (per investigator) as defined by RECIST v1.1,
OS,
ORR, DOR, and PFS as defined by RECIST v1.1 via blinded independent central review
(BICR),
PK parameters (including, but not limited to AUC, Cₘₐₓ, Tₘₐₓ, half-life) of Compound
(10b) and nivolumab from plasma or serum concentration-time data,
Pharmacodynamic parameters (including but not limited to raw, normalized, and/or
baseline adjusted analyte signal in circulating and intratumoral target engagement
biomarkers of Compound (10b) activity in combination with nivolumab)
Measurement of ADA to nivolumab, and
Incidence and severity of TEAEs and SAEs.

Biomarkers that correlate with response to treatment will be an exploratory endpoint.
Statistical Considerations: Descriptive statistics will be employed to analyze the data.
Summary statistics for continuous variables will include the mean, standard deviation, median,
and range (minimum and maximum); categorical variables will be presented as frequency counts
and percentages; and time-to-event variables will be summarized by Kaplan-Meier medians and
curves.
Pharmacokinetic parameters of Compound (10b) and nivolumab will be determined based on
individual plasma or serum concentration-time profiles using noncompartmental methods.
Efficacy analyses will be conducted on the intent-to-treat (ITT) population, unless otherwise
specified. Efficacy data (e.g., PFS, ORR, DOR) will be summarized using descriptive statistics
and presented by study part, dose level, as appropriate.
Efficacy data are considered to be descriptive; no formal statistical testing will be performed.
Adverse events will be coded using the Medical Dictionary for Regulatory Activities.
Tabulations of TEAEs will be prepared by study part, dose level, as appropriate. Treatment-
emergent AEs will be defined as AEs that first occur or worsen in severity after the first
administration of study treatment through 100 days after the last administration of study
treatment. Serious adverse events (SAEs) will be collected from the time of signing the informed
consent through 100 days after the last administration of study treatment. Clinical laboratory test
results, vital signs, ECGs, and multiple-gated acquisition scan (MUGA)/echocardiogram
(ECHO) results will be summarized appropriately.

TABLE 1
Schedule of Assessments: Phase 1a Dose Escalation and Phase 1b Dose Expansion

| | | | | | | | | | | Subsq | Subsq | | | | |
|---|---|---|---|---|---|---|---|---|---|---|---|---|---|---|---|
| | | | | | | | | | | ODD | EVEN | | | | |
| | | | | | | | | | | Cycles | Cycles | | | | Long- |
| | | Screen- | | | | | | | | (eg, 3, | (eg, 4, | | EOT | EOT | Term |
| | | ing | Cycle 1 | | | | | Cycle 2^{CC} | | 5)^{CC} | 6)^{CC} | ET | FU V1 | FU V2 | FU |
| | | Study Day ^{A} | | | | | | | | | | | | | |
| | | | | | | | | | | | | | Last | Last | Every |
| | | −30 | | | | | | | | | | | Dose +28 | Dose +100 | 3 |
| | | to −1 | 1 ^{U, V} | 2 | 8 | 15 | 22^{KK} | 1 ^{U} | 15 | 1 ^{U} | 1 ^{U} | NA | d | d | months |
| | Pre- | +/− days allowable ^{A} | | | | | | | | | | | | | |
| | Screen- | | | | | | −1 | | | | | | | | |
| | ing | 0 | 0 | 0 | ±3 | ±3 | to ±3 | ±3 | ±3 | | ±3 | NA | ±3 | ±5 | NA |
| Molecular | | | | | | | | | | | | | | | |
| Pre- | | | | | | | | | | | | | | | |
| screening ^{LL} | | | | | | | | | | | | | | | |
| Informed | X | | | | | | | | | | | | | | |
| consent | | | | | | | | | | | | | | | |
| Testing | X | | | | | | | | | | | | | | |
| Study | | | | | | | | | | | | | | | |
| Consent | | | | | | | | | | | | | | | |
| Informed | | X | | | | | | | | | | | | | |
| consent | | | | | | | | | | | | | | | |
| Confirmation | | | | | | | | | | | | | | | |
| of Disease | | | | | | | | | | | | | | | |
| State^{D} | | | | | | | | | | | | | | | |
| Advanced | | X | | | | | | | | | | | | | |
| NSCLC | | | | | | | | | | | | | | | |
| Confirmation | | | | | | | | | | | | | | | |
| of Tumor | | | | | | | | | | | | | | | |
| Mutation ^{C} | | | | | | | | | | | | | | | |
| Documented | | X | | | | | | | | | | | | | |
| KRAS | | | | | | | | | | | | | | | |
| mutation | | | | | | | | | | | | | | | |
| Administration | | | | | | | | | | | | | | | |
| of Study | | | | | | | | | | | | | | | |
| Treatment ^{B} | | | | | | | | | | | | | | | |
| Compound (10b) | | | X-------------------------------------------------------------- | | | | | | | | | | | | |
| | | | --------------------------X | | | | | | | | | | | | |
| Nivolumab | | | X | | | | | X | | X | X | | | | |
| Reconciliation | | | | | X | X | X | X | X | X | X | X | X | | |
| between | | | | | | | | | | | | | | | |
| patient diary | | | | | | | | | | | | | | | |
| and pill | | | | | | | | | | | | | | | |
| count | | | | | | | | | | | | | | | |
| DLT | | | | | | | | | | | | | | | |
| Assessment | | | | | | | | | | | | | | | |
| (Phase | | | | | | | | | | | | | | | |
| 1a Dose | | | | | | | | | | | | | | | |
| Escalation | | | | | | | | | | | | | | | |
| Only) | | | | | | | | | | | | | | | |
| DLT | | | X----------------------------------------X | | | | | | | | | | | | |
| Assessment | | | | | | | | | | | | | | | |
| Period ^{PP} | | | | | | | | | | | | | | | |
| Disease | | | | | | | | | | | | | | | |
| Assessment | | | | | | | | | | | | | | | |
| Restaging | | X | | | | | | | | X | | | X | | |
| scan (CT, | | | | | | | | | | (±7 d) | | | (±7 d)^{HH} | | |
| MRI, or | | | | | | | | | | | | | | | |
| PET-CT) ^{E} | | | | | | | | | | | | | | | |
| Relevant | | X | | | | | | | | X | | | X | | |
| tumor | | | | | | | | | | | | | | | |
| marker ^{F} | | | | | | | | | | | | | | | |
| Study | | | | | | | | | | | | | | | |
| Procedures & | | | | | | | | | | | | | | | |
| Examinations | | | | | | | | | | | | | | | |
| Eligibility ^{G} | | X^{G} | | | | | | | | | | | | | |
| Demographics ^{H} | | X | | | | | | | | | | | | | |
| Medical | | X | X^{S} | | | | | | | | | | | | |
| history ^{I} | | | | | | | | | | | | | | | |
| ECG | | X | X^{W, GG} | X^{Z} | | | | X^{W, GG} | | X^{FF, GG} | | X | X | X | |
| MUGA/ECHO | | X | | | | | | X^{DD} | | X^{DD} | | X^{DD, II} | X^{DD, II} | | |
| Physical | | X | X^{S} | | X | | | X | | X | X | X | X | X | |
| Examination ^{J} | | | | | | | | | | | | | | | |
| ECOG | | X | X^{X, Y} | | | | | X | | X | X | X | X | X | |
| performance | | | | | | | | | | | | | | | |
| status | | | | | | | | | | | | | | | |
| Vital | | X | X^{Y} | X^{Z} | X^{Y} | X^{Y} | | X^{Y} | X^{Y} | X^{Y} | X^{Y} | X | X | X | |
| signs ^{K} | | | | | | | | | | | | | | | |
| Prior/ | | Document any medication or vaccine that the patient is receiving at the time of | | | | | | | | | | | | | |
| Concomitant | | signing the ICF and/or within 30 d or 5 half-lives (whichever is longer) of first | | | | | | | | | | | | | |
| medica- | | dose of study treatment. Document concomitant medications taken from time of signing | | | | | | | | | | | | | |
| tions^{MM} | | the ICF through 100 d after the last dose of study treatment or date of the | | | | | | | | | | | | | |
| | | decision to permanently discontinue study treatment. | | | | | | | | | | | | | |
| AE | SAEs | SAEs | Document AEs from first dose of study treatment through at least 100 d | | | | | | | | | | | | |
| reporting ^{L} | only | only | after last dose of study treatment. AE information will be collected at | | | | | | | | | | | | |
| | | | study visits or over the phone. | | | | | | | | | | | | |
| Survival | | | | | | | | | | | | | | | X ^{JJ} |
| Anticancer | | | | | | | | | | | | | | | X ^{JJ} |
| therapies | | | | | | | | | | | | | | | |
| since drug | | | | | | | | | | | | | | | |
| discontin- | | | | | | | | | | | | | | | |
| uation | | | | | | | | | | | | | | | |
| Laboratory | | | | | | | | | | | | | | | |
| Evaluations ^{M} | | | | | | | | | | | | | | | |
| FSH ^{N} | | X | | | | | | | | | | | | | |
| Pregnancy | | X | X | | | | | X | | X | X | X | X | X | |
| test ^{O} | | | | | | | | | | | | | | | |
| HIV, | | X | | | | | | | | | | | | | |
| HBV & HCV | | | | | | | | | | | | | | | |
| Blood | | X | X^{Y} | X^{Z} | X^{Y} | X^{Y} | | X^{Y} | X^{Y} | X^{Y} | X^{Y} | X | X | X | |
| hematology ^{P} | | | | | | | | | | | | | | | |
| Chemistry ^{Q} | | X | X^{Y} | X^{Z} | X^{Y} | X^{Y} | | X^{Y} | X^{Y} | X^{Y} | X^{Y} | X | X | X | |
| Coagulation | | X | X^{W} | X^{Z} | | | | X^{W} | | X^{Y} | X^{Y} | X | X | X | |
| (aPTT, PT, | | | | | | | | | | | | | | | |
| INR) | | | | | | | | | | | | | | | |
| Urinalysis | | X | X^{Y} | | | | | X^{Y} | | X^{Y} | X^{Y} | X | X | X | |
| (spot | | | | | | | | | | | | | | | |
| collection) | | | | | | | | | | | | | | | |
| Research | | | | | | | | | | | | | | | |
| Studies: | | | | | | | | | | | | | | | |
| PK/ | | | | | | | | | | | | | | | |
| Pharmaco- | | | | | | | | | | | | | | | |
| dynamic ^{NN} | | | | | | | | | | | | | | | |
| Comp. | | | X^{AA} | X^{Z} | X^{Y} | X^{Y} | | X^{AA} | | X^{FF} | X^{FF} | | | | |
| (10b) PK | | | | | | | | | | | | | | | |
| samples | | | | | | | | | | | | | | | |
| Nivolumab | | | X^{AA} | | | | | X^{AA} | | | X^{OO} | | | | |
| PK samples | | | | | | | | | | | | | | | |
| Nivolumab | | | X^{Y} | | | | | X^{Y} | | | X^{OO} | | | | |
| ADA | | | | | | | | | | | | | | | |
| samples | | | | | | | | | | | | | | | |
| Pharmaco- | | | X^{BB} | X^{Z} | | | | X^{BB} | | X^{FF} | X^{FF} | | X | | |
| dynamic - | | | | | | | | | | | | | | | |
| Blood/ | | | | | | | | | | | | | | | |
| PBMC | | | | | | | | | | | | | | | |
| samples | | | | | | | | | | | | | | | |
| Circulating | | X | | | | | | X | | X | X | | X | | |
| tumor | | | | | | | | | | | | | | | |
| markers ^{R} | | | | | | | | | | | | | | | |
| Fresh | | X^{T} | | | | | | X^{EE} | | | | | X | | |
| tumor | | | | | | | | | | | | | (±7 d) | | |
| biopsy | | | | | | | | | | | | | | | |

### Abbreviations and Footnotes of Table 1

Abbreviations. ADA=anti-drug antibody; AE=adverse event; aPTT=activated partial thromboplastin time; CR=complete response(s); CT=computed tomography; ctDNA=circulating tumor deoxyribonucleic acid; ECG=electrocardiogram; ECHO=echocardiogram; ECOG=Eastern Cooperative Oncology Group; EOS=End of study; EOT=End of treatment; ET=Early Termination; DLT=dose-limiting toxicity; FDA=Food and Drug Administration; FSH=follicle-stimulating hormone; FU=Follow-Up; HBV=hepatitis B virus; HCV=hepatitis C virus; HIV=human immunodeficiency virus; ICF=informed consent form; INR=international normalized ratio; IV=intravenous; KRAS=Kirsten rat sarcoma viral oncogene homolog; LVEF=left ventricular ejection fraction; MRI=magnetic resonance imaging; MUGA=multiple-gated acquisition scan; NA=not applicable; NSCLC=non-small cell lung cancer; PBMC=peripheral blood mononuclear cell; PD=progressive disease; PET=positron emission tomography; PK=pharmacokinetic; PR=partial response; PT=prothrombin time; QD=once daily; QTcF=QT using Fridericia's correction formula; RECIST=Response Evaluation Criteria in Solid Tumor; RP2D=recommended phase 2 dose.
^{A.} Due to the Coronavirus Disease 2019 (COVID-19) pandemic, telephone or video contact visits for safety monitoring, alteration of the visit schedule, or other appropriate clinical trial conduct changes, may occur. The study days allowable are intended as a best practice; however, may be adjusted as required. If necessary, additional changes to the schedule of events will be performed and documented consistent with current FDA guidance (e.g., FDA Guidance of Clinical Trials of Medical Products during COVID-19 Public Health Emergency).
^{B.} In each 28-day cycle, Compound (10b) will be taken orally QD, in combination with nivolumab administered IV once every 4 wk (28 d) via IV infusion. The RP2D selected from Phase 1a Dose Escalation will be used for Compound (10b) in Phase 1b Dose Expansion. The dose may be adjusted based on an SRC decision following review of the totality of the data. The nivolumab infusion should be administered approximately 30 minutes after administration of Compound (10b). Compound (10b) should be taken with approximately 240 mL (8 oz) of water after an overnight fast (minimum 8 hr) followed by 2 hr of fasting after the dose is taken.
^{C.} Confirm documentation of a KRAS mutation from local or central laboratory testing in tumor samples collected within the 1 yr prior to screening. Note: Determination of KRAS mutation status by the central laboratory as confirmation of local laboratory testing is not required.
^{D.} All patients must have progression or disease recurrence on or after 2^{nd} line+therapy.
^{E.} Restaging scans (CT, MRI, or PET-CT) should be done at screening, C3D1 (±7 d) and then every 8 wk (±7 d) thereafter until PD. Scans are to be taken at the EOT Follow-up Visit 1 unless taken within 28 d of the visit. Confirmatory scans will also be obtained at least 4 wk following initial documentation of an objective response (i.e., PR or CR per RECIST v1.1). The type of scan obtained is at the discretion of the investigator as appropriate for the disease. However, the same method should be used for the duration of the study. All scans will be read locally; in Phase 1b Dose Expansion, scans will also be read via blinded independent central review.
^{F.} Relevant tumor markers will be assessed as appropriate using blood samples.
^{G.} Eligibility to be confirmed prior to dosing.
^{H.} Including year of birth, sex, height, race, ethnicity, as allowed per regional regulations.
^{I.} Including relevant medical history, current medical conditions, oncology history (e.g., diagnosis, extent of cancer and stage, prior anticancer therapy), radiation history.
^{J.} Complete physical examination to be performed at screening and EOT Follow-up Vi or ET (as applicable); focused examination (e.g., symptom directed per investigator's judgment) to be performed at other visits; includes weight for both complete and focused examinations.
^{K.} Vital signs should be taken before any blood draw.
^{L.} SAEs should be collected from time of informed consent. Death solely due to progression of disease should not be reported as an SAE but as with all deaths, must be captured on the relevant CRF page.
^{M.} Blood hematology, chemistry, coagulation, pregnancy, and urinalysis pre-dose assessments may be performed up to 2 d prior to scheduled visit. If these screening assessments are performed within 48 hr prior to Cycle 1 Day 1, these results may be used as baseline (pre-dose assessments) without requiring a repeat assessment.
^{N.} Women who are not surgically sterile to confirm menopausal state.
^{O.} Women who are not surgically sterile or menopausal with serum test at all indicated time points. Patients must have a negative test within 48 hr of D1 of every cycle.
^{P.} Complete blood count, differential, platelets.
^{Q.} See Section 8.2.2 of the clinical protocol for the list of parameters in the clinical chemistry panel [NOTE: patient should fast for all screening labs and all predose labs]
^{R.} Including ctDNA. Samples should be collected after at least 7 consecutive days of Compound (10b) dosing.
^{S.} Medical history and physical exam should be confirmed and updated with any findings which may have occurred prior to the first dose on C1D1.
^{T.} Fresh tumor biopsies should be collected for all patients enrolled in the study if at all feasible. If fresh tumor biopsies were not done during molecular prescreening and are not feasible during Screening, the most recent archival tumor biopsies collected preferably within 6 months but no later than 1 yr of enrollment and with sufficient tumor for central testing of KRAS mutations can be used. Patients may be enrolled even when tumor biopsies cannot be obtained at the discretion of the medical monitor, in consultation with the investigator. Enrollment of patients for whom collection of tumor biopsies will not occur will be handled on a case-by-case basis by the medical monitor. Please refer to the Lab Specimen Manual for details regarding collection and handling of tumor tissue samples.
^{U.} If the start of the sequent cycle is delayed due to simultaneous dose interruptions of both study medications, weekly assessments should be recorded as unscheduled visits to the just completed 28-d cycle until the dosing of at least one study treatment is resumed. The first day of the next cycle will be the day a study treatment is restarted. Should either Compound (10b) be interrupted for 15 consecutive days or nivolumab for 12 weeks, the patient should be discontinued permanently from both study medications.
^{V.} Cycle 1 Day 1 visit should take place on a Monday, Tuesday, Wednesday, or Thursday.
^{W.} Collect pre-dose and 4 hr (all time points±15 min) after administration of Compound (10b).
^{X.} Unless done in the prior 7 d.
^{Y.} Collect pre-dose within 2 hr prior to dosing of Compound (10b) or nivolumab if Compound (10b) dosing is on hold on day of visit.
^{Z.} Collect pre-dose (i.e., approximately 24 hr [±2 hr] after prior day's administration of Compound (10b) but before dosing of Compound (10b) on day of visit).
^{AA.} For Compound (10b) PK: Collect blood samples pre-dose (within 2 hr prior to dosing of Compound (10b)) and 0.5 (+5 min), 1 (+10 min), 2 (+15 min), 4 (+15 min) and 6 hr (±15 min) after administration of Compound (10b). Steady-state (C2D1) PK samples should be collected after at least 7 consecutive days of dosing of Compound (10b). For Nivolumab PK: Collect blood samples pre-dose (within 2 hr prior to dosing of Compound (10b) or nivolumab if Compound (10b) dosing is on hold) and at end of infusion (within 5 min prior to end of infusion)
^{BB.} Collect pre-dose (within 2 hr prior to dosing of Compound (10b) or nivolumab if Compound (10b) dosing is on hold) and 2 (±15 min) and 4 hr (+15 min) after administration of Compound (10b) (or nivolumab if Compound (10b) dosing is on hold).
^{CC.} If the patient will not be continuing on subsequent cycle (i.e., not continuing on study), the patient should still undergo all Day 1 visit procedures of the subsequent cycle, except the 2 hr PK and pharmacodynamic samples.
^{DD.} Window (+3 d) allowed for each MUGA/ECHO evaluation starting at Cycle 2.
^{EE.} Collect post-dose between 2 and 6 hr after administration of Compound (10b) or nivolumab if Compound (10b) dosing is on hold on Cycle 2 Day 1 (+7 d)
^{FF.} Collect pre-dose within 2 hr prior to and 2 hr (+15 min) after administration of Compound (10b) on day of visit. Samples should be collected after at least 7 consecutive days of Compound (10b) dosing.
^{GG.} The 12-lead ECG will be taken in triplicate at screening and at C1D1, C2D1, C3D1, and every 2 cycles thereafter.
^{HH.} Unless completed within 4 wk of the EOT Follow-up Visit 1.
^{II}. A MUGA scan or ECHO will be performed at the EOT Follow-up Visit 1 and the ET visit only if an assessment of LVEF has not been performed within the last 14 d.
^{JJ.} Patients who discontinue treatment for radiographic PD as well as patients who discontinue treatment for reasons other than PD and subsequently have radiographic PD will be followed approximately every 3 months (via telephone or office visit) for survival status and new anticancer therapy information until EOS. This survival follow-up also applies to patients who discontinue study treatment for reasons other than radiographic PD but withdraw consent for further tumor assessments. Survival follow-up will continue until all patients have discontinued study treatment, died, withdrawn consent, or been lost to follow-up, or until EOS, whichever occurs first. Long-term follow up will also include the collection of disease response(s) and secondary malignancies.
^{KK.} C1D22 will be a telephone visit during which the daily self-administration of Compound (10b) will be reviewed, per the patient diary, and information on AEs and concomitant medications will be recorded in source documents and transcribed to the CRF.
^{LL.} Only if there is no documentation of KRAS mutation within the 1 yr prior to screening, molecular prescreening for KRAS mutation using an appropriate clinically validated and/or FDA approved test
^{MM.} Concomitant medications include all prescription and nonprescription medication (over-the-counter or prescription), vaccine, or dietary supplement (vitamins, minerals, herbs).
^{NN.} For details on the pharmacodynamic assessments, refer to Section 8.5 of the clinical protocol.
^{OO.} Collect blood samples pre-dose (within 2 hr prior to dosing of Compound (10b) or nivolumab if Compound (10b) dosing is on hold) at C4D1 and then every 4 cycles thereafter (e.g., C8D1, C12D1).
^{PP.} To qualify for the DLT assessment period, patients need to have taken 21 doses within the 28-day assessment period. See Section 9.1.1 of the clinical protocol for definition of DLT.

TABLE 2
Planned Phase 1a Dose Escalation

| | |
|---|---|
| Dose Level/Number | Planned Doses |

| of Patients^{a} | Compound (10b)^{b} | Nivolumab |
|---|---|---|
| Level 1 (n = 5-7) | 250 mg QD | 480 mg every 4 wk |
| Level 2 (n = 5-7) | 400 mg QD | 480 mg every 4 wk |
| Level 3 (n = 5-7) | 550 mg QD | 480 mg every 4 wk |

^{a}Additional patients may be enrolled in existing cohorts if deemed necessary by the SRC to evaluate the safety, tolerability, and the RP2D.
^{b}Dose escalation, an additional dose level, alternative dose levels, and/or alternative dose schedules may be enrolled following SRC review of the totality of the data and after the SRC approves or makes a recommendation regarding the next dose cohort.
Abbreviations:
QD = once daily;
RP2D = recommended phase 2 dose;
SRC = Safety Review Committee;
wk = week

DLT is defined as an AE or abnormal laboratory value, excluding toxicities clearly related to disease progression or intercurrent illness, and occurring during the first cycle (28 days) on study that meets any of the following criteria:
Grade 3 laboratory abnormalities lasting more than 7 days or Grade 4 laboratory abnormalities of aspartate amino transferase/alanine amino transferase (AST/ALT) and/or bilirubin, of any duration, with the following exceptions:
   For patients with Grade 1 AST/ALT at baseline (>ULN to 3×ULN), an AST/ALT value of >7.5×ULN will be considered a DLT
   For patients with Grade 2 AST/ALT at baseline (>3×ULN to >5×ULN), an AST/ALT value >10×ULN will be considered a DLT
Grade 3 or higher non-hematological toxicity excluding:
   Grade 3 nausea, vomiting or diarrhea for less than 72 hours with adequate supportive care
   Grade 3 fatigue lasting less than a week
   Grade 3 electrolyte abnormality that lasts for less than 72 hours that is not clinically complicated and resolves spontaneously or with conventional medical interventions
   Grade 3 amylase or lipase lasting less than 72 hours and not associated with manifestations of pancreatitis
Meet criteria for Hy's Law (i.e., ≥3×ULN ALT and/or AST with ≥2×ULN total bilirubin and alkaline phosphatase <2×ULN), unless patient has congenital hyperbilirubinemia.
Grade 3 neutropenia >7 days.
Grade 4 neutropenia.
Febrile neutropenia [ANC <1000/mm³ with single oral temperature >38.3° C. (101° F.) or with two consecutive oral temperatures ≥38° C. (100.4° F.) measured at least 1 hour apart].
Grade 3 thrombocytopenia with Grade 2 or higher bleeding.
Grade 4 thrombocytopenia or thrombocytopenia requiring platelet transfusion.
Grade 4 life-threatening anemia.
Grade 5 toxicities.
Any other ≥Grade 3 clinically significant or persistent toxicity that may be considered a DLT following review by the SRC.

### Appendix 1 ECOG Performance Status

| Grade | ECOG Performance Status |
|---|---|
| 0 | Fully active, able to carry on all pre-disease performance without restriction |
| 1 | Restricted in physically strenuous activity but ambulatory and able to carry out |
| | work of a light or sedentary nature, e.g., light house work, office work |
| 2 | Ambulatory and capable of all selfcare but unable to carry out any work |
| | activities. Up and about more than 50% of waking hours |
| 3 | Capable of only limited selfcare, confined to bed or chair more than 50% of |
| | waking hours |
| 4 | Completely disabled. Cannot carry on any selfcare. Totally confined to bed or |
| | chair |
| 5 | Dead |

Oken M M, Creech R H, Tormey D C, et al. Toxicity and response criteria of the Eastern Cooperative Oncology Group. Am J Clin Oncol 1982; 5(6): 649-655.

### Appendix 2 List of Highly Effective Methods of Contraception

The following is from the Clinical Trials Facilitation Group 2014.

Methods that can achieve a failure rate of less than 1% per year when used consistently and correctly are considered as highly effective birth control methods. Such methods include:
combined (estrogen and progestogen containing) hormonal contraception associated with
inhibition of ovulation¹:
oral
intravaginal
transdermal
progestogen-only hormonal contraception associated with inhibition of ovulation 1:
oral
injectable
implantable²
intrauterine device (IUD)²
intrauterine hormone-releasing system (IUS)²
bilateral tubal occlusion²
vasectomised partner^{2,3}
sexual abstinence ⁴
1. Hormonal contraception may be susceptible to interaction with the IMP, which may reduce the efficacy of the contraception method (see Clinical Trials Facilitation Group 2014, Section 4.3).
2. Contraception methods that in the context of this guidance are considered to have low user dependency.
3. Vasectomized partner is a highly effective birth control method provided that partner is the sole sexual partner of the WOCBP trial patient and that the vasectomized partner has received medical assessment of the surgical success.
4. In the context of this guidance sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the patient.

### Appendix 3 List of Excluded Medications, Herbal Supplements and Food Products as they Pertain to Compound (10b)

| | | | Drugs Potentially Affected | | |
|---|---|---|---|---|---|
| | Drugs Potentially | | by Coadministration | | |
| | Affecting Exposure | | of Compound (10b) | | |
| | of Compound (10b) | | MATE1/2-K | P-gp | BCRP |
| Medication | CYP3A4 | P-gp | Substrates | Substrates | Substrates |
| Aliskiren | | | | X | |
| Alogliptin | | Inhibitor | | | |
| Ambrisentan | | | | X | |
| Amiodarone | | Inhibitor | | | |
| Amprenavir | Inhibitor (M) | | | | |
| Apalutamide | Inducer (S) | Inducer | | | |
| Apixaban | | | | X | |
| Apremilast | | | | X | |
| Aprepitant | Inhibitor (M) | | | X | |
| Asian ginseng | | Inhibitor | | | |
| (Panax ginseng) | | | | | |
| Asunaprevir | | Inhibitor | | | |
| Atazanavir | Inhibitor (M) | | | X | |
| Atazanavir/Ritonavir | Inhibitor (M) | | | | |
| Atorvastatin | | | | X | X |
| Avasimibe | Inducer (S) | Inducer | | | |
| Avatrombopag | | | | X | |
| Azithromycin | | Inhibitor | | | |
| Baricitinib | | | X | X | X |
| Betrixaban | | | | X | |
| Boceprevir | Inhibitor (S) | | | X | |
| Bosentan | Inducer (M) | | | | |
| Bosutinib | | | | X | |
| Budesonide | | | | X | |
| Canaglifozin | | Inhibitor | | X | |
| Capmatinib | | Inhibitor | | | |
| Captopril | | Inhibitor | | | |
| Carbamazepine | Inducer (S) | Inducer | | | |
| Carvedilol | | Inhibitor | | X | |
| Casopitant | Inhibitor (M) | | | | |
| Ceritinib | Inhibitor (S) | | | | |
| Cimetidine | Inhibitor (M) | Inhibitor | | | |
| Ciprofloxacin | Inhibitor (M) | | | | |
| Clarithromycin | Inhibitor (S) | Inhibitor | | | |
| Clobazam | | | | X | |
| Clopidogrel | | Inhibitor | | | |
| Cobicistat | Inhibitor (S) | Inhibitor | | | |
| Cobimetinib | | | | X | |
| Conivaptan | Inhibitor (M) | Inhibitor | | X | |
| Coumestrol | | | | | X |
| Cremophor EL | | Inhibitor | | | |
| Cremophor RH | | Inhibitor | | | |
| Crizotinib | Inhibitor (M) | | | X | |
| Cyclosporine | Inhibitor (M) | | | X | |
| Dabigatran etexilate | | | | X | |
| Dabrafenib | Inducer (M) | | | X | |
| Daclatasvir | | Inhibitor | | X | |
| Danoprevir/Ritonavir | Inhibitor (S) | | | | |
| Dan-shen | | Inducer | | | |
| (Salvia miltiorrhiza) | | | | | |
| Dantrolene | | | | | X |
| Darolutamide | | | | X | |
| Darunavir | Inhibitor (M) | | | | |
| Darunavir/Ritonavir | Inhibitor (M) | | | | |
| Digoxin | | | | X | |
| Diltiazem | Inhibitor (M) | Inhibitor | | X | |
| Diosmin | | Inhibitor | | | |
| Dolutegravir | | | | X | X |
| Doxorubicin | | | | X | |
| Dronedarone | Inhibitor (M) | Inhibitor | | | |
| Duvelisib | Inhibitor (M) | | | X | X |
| Edoxaban | | | | X | |
| Efavirenz | Inducer (M) | Inducer | | | |
| Elagolix | Inducer (M) | Inhibitor | | | |
| Elexacaftor | | | | X | |
| Eliglustat | | Inhibitor | | X | |
| Elvitegravir/Ritonavir | Inhibitor (S) | | | | |
| Enzalutamide | Inducer (S) | | | | |
| Erythromycin | Inhibitor (M) | Inhibitor | | | |
| Estrone sulfate | | | | | X |
| Etravirine | Inducer (M) | | | | |
| Everolimus | | | | X | |
| Faldaprevir | Inhibitor (M) | | | | |
| Fedratinib | Inhibitor (M) | | | X | |
| Felodipine | | Inhibitor | | | |
| Fexofenadine | | | | X | |
| Five-flavor berry | | Inhibitor | | | |
| Fidaxomicin | | | | X | |
| Fluconazole | Inhibitor (M) | | | | |
| Fluvoxamine | Inhibitor (M) | Inhibitor | | | |
| Fostamatinib | | Inhibitor | | | |
| Genistein | | Inducer | | | X |
| Gilteritinib | | | | X | |
| Ginkgo (Ginkgo biloba) | | Inhibitor | | | |
| Glasdegib | | | | X | X |
| Glecaprevir/Pibrentasvir | | Inhibitor | | X | X |
| Grapefruit juice | Inhibitor (S) | | | | |
| Grapefruit juice DS | Inhibitor (S) | | | | |
| Green tea | | Inducer | | | |
| Idelalisib | Inhibitor (S) | | | X | |
| Imatinib | Inhibitor (M) | | | X | X |
| Indacaterol | | | | X | |
| Indinavir | Inhibitor (S) | | | X | X |
| Indinavir/Ritonavir | Inhibitor (S) | Inhibitor | | | |
| Irinotecan | | | | X | X |
| Isavuconazole | Inhibitor (M) | Inhibitor | | | |
| Istradefylline | Inhibitor (M) | Inhibitor | | | |
| Itraconazole | Inhibitor (S) | Inhibitor | | | |
| Ivacaftor | | Inhibitor | | | |
| Ivosidenib | Inducer (S) | | | X | |
| Ketoconazole | Inhibitor (S) | Inhibitor | | | |
| Lapatinib | | Inhibitor | | X | |
| Larotrectinib | | | | X | X |
| Ledipasvir | | | | X | X |
| Lefamulin | Inhibitor (M) | | | X | |
| Lenvatinib | | | | X | X |
| Lersivirine | Inducer (M) | | | | |
| Lesinurad | Inducer (M) | | | | |
| Letermovir | Inhibitor (M) | | | | |
| Linagliptin | | | | X | |
| Loperamide | | | | X | |
| Lopinavir | Inducer (M) | | | X | |
| Lopinavir/Ritonavir | Inhibitor (S) | Inhibitor | | | |
| Lorlatinib | Inducer (M) | | | | |
| Lovastatin | | | | X | |
| Lumacaftor | Inducer (S) | | | | |
| Magnolia vine | Inhibitor (M) | | | | |
| (Schisandra | | | | | |
| sphenanthera) | | | | | |
| Metformin | | | X | | |
| Methotrexate | | | | | X |
| Methylprednisolone | | | | X | |
| Mibefradil | Inhibitor (S) | Inhibitor | | | |
| Mifepristone | Inhibitor (S) | Inhibitor | | | |
| Milk thistle | | Inhibitor | | | |
| (Silybum marianum) | | | | | |
| Mirabegron | | Inhibitor | | | |
| Mitotane | Inducer (S) | | | | |
| Modafinil | Inducer (M) | | | | |
| Nafcillin | Inducer (M) | | | | |
| Nefazodone | Inhibitor (S) | Inhibitor | | | |
| Nelfinavir | Inhibitor (S) | Inhibitor | | X | |
| Neratinib | | Inhibitor | | | |
| Netupitant | Inhibitor (M) | | | | |
| Nifedipine | | Inhibitor | | | |
| Nilotinib | Inhibitor (M) | | | | |
| Nintedanib | | | | X | |
| Nitrendipine | | Inhibitor | | | |
| Osimertinib | | Inhibitor | | | |
| Ozanimod | | | | | X |
| Paclitaxel | | | | X | |
| Panobinostat | | | | X | |
| Paritaprevir/Ritonavir/ | Inhibitor (S) | Inhibitor | | | |
| Ombitasvir | | | | | |
| Paroxetine | | Inhibitor | | X | |
| Pexidartinib | Inducer (M) | Inhibitor | | | |
| Phenobarbital | Inducer (S) | | | | |
| Phenytoin | Inducer (S) | Inducer | | | |
| Piperine | | Inhibitor | | | |
| Pitavastatin | | | | X | X |
| Posaconazole | Inhibitor (S) | | | X | |
| Pralsetinib | | | | X | |
| Prazosin | | | | X | X |
| Primidone | Inducer (M) | | | | |
| Propafenone | | Inhibitor | | | |
| Quercetin | | Inhibitor | | | |
| Quinidine | | Inhibitor | | X | |
| Quinine | | Inhibitor | | | |
| Ranolazine | | Inhibitor | | | |
| Ravuconazole | Inhibitor (M) | | | | |
| Ribociclib | Inhibitor (S) | | | | |
| Rifabutin | Inducer (M) | Inducer | | | |
| Rifampin | Inducer (S) | Inhibitor | | | |
| Rifapentine | Inducer (S) | | | | |
| Rimegepant | | | | X | X |
| Riociguat | | | | X | X |
| Risperidone | | | | X | |
| Ritonavir | Inhibitor (S) | Inhibitor | | X | |
| Rivaroxaban | | | | X | |
| Rolapitant | | Inhibitor | | | |
| Romidepsin | | | | X | |
| Rosuvastatin | | | | | X |
| Rucaparib | | Inhibitor | | | |
| Saquinavir | Inhibitor (S) | | | X | |
| Saquinavir/Ritonavir | Inhibitor (S) | Inhibitor | | | |
| Sarecycline | | Inhibitor | | | |
| Selexipag | | | | X | |
| Semagacestat | Inducer (M) | | | | |
| Seville oranges | Inhibitor (M) | Inhibitor | | | |
| Silodosin | | | | X | |
| Simeprevir | | Inhibitor | | | |
| Simvastatin | | | | X | |
| Sirolimus | | | | X | |
| Sofosbuvir | | | | X | X |
| Sofosbuvir/Velpatasvir/ | | Inhibitor | | | |
| Voxilaprevir | | | | | |
| Star fruit | Inhibitor (S) | | | | |
| St. John's Wort Extract | Inducer (S) | Inhibitor | | | |
| Sulfasalazine | | | | | X |
| Suvorexant | | Inhibitor | | | |
| Tacrolimus | | | | X | |
| Talazoparib | | | | X | X |
| Talinolol | | Inhibitor | | X | |
| Talviraline | Inducer (M) | | | | |
| Telaprevir | Inhibitor (S) | Inhibitor | | X | |
| Telithromycin | Inhibitor (S) | Inhibitor | | | |
| Telmisartan | | Inhibitor | | | |
| Telotristat ethyl | Inducer (M) | | | | |
| Tenofovir alafenamide | | | | X | X |
| Tezacaftor | | | | X | |
| Thioridazine | Inducer (M) | | | | |
| Ticagrelor | | Inhibitor | | | |
| Tipranavir | | | | X | |
| Tipranavir/Ritonavir | Inhibitor (S) | Inhibitor | | | |
| Tivantinib | | Inducer | | | |
| Tofisopam | Inhibitor (M) | | | | |
| Tolvaptan | | Inhibitor | | | |
| Topotecan | | | | X | X |
| Troleandomycin | Inhibitor (S) | | | | |
| Tucatinib | Inhibitor (S) | Inhibitor | | | |
| Ubrogepant | | | | X | X |
| Umeclidinium | | | | X | |
| Valbenazine | | Inhibitor | | | |
| Valspodar (PSC 833) | | Inhibitor | | | |
| Vandetanib | | Inhibitor | | | |
| Velpatasvir | | Inhibitor | | X | X |
| Vemurafenib | | Inhibitor | | | |
| Venetoclax | | Inhibitor | | X | X |
| Verapamil | Inhibitor (M) | Inhibitor | | X | |
| Viekira Pak | Inhibitor (S) | | | | |
| Vinblastine | | | | X | |
| Vincristine | | | | X | |
| Voclosporin | | Inhibitor | | X | |
| Vorapaxar | | Inhibitor | | | |
| Voriconazole | Inhibitor (S) | | | | |
| Voxilaprevir | | | | X | X |
| Zanubrutinib | | Inhibitor | | | |

### Appendix 4 List of Medications to be Used with Caution in Combination with Compound (10b)

OATP1B1 and/or OATP1B3 Substrates
Cholescystokinin
Eluxadoline
Estradiol-glucuronide
Fluvastatin
Glyburide
Nateglinide
Pitavastatin
Pravastatin
Repaglinide

### Example 3: A Phase 1 Study of the SHP2 Inhibitor Compound (10b) in Combination with Nivolumab in Patients with Advanced Non-Small Cell Lung Cancer with a KRAS Mutation—Dosing Adjustments, Delays, and Discontinuations

### a. Compound (10b)

Compound (10b) dose modification decisions should be based on the CTCAE grading scale v5.0 and according to Table 3. If drug-related AEs are not specified in the table, doses may be reduced or held or permanently discontinued at the discretion of the investigator for the patient's safety.

For each patient, a maximum of two dose reductions of Compound (10b) will be allowed. Dose reduction for Compound (10b) means treatment at the next lower, previously tested dose level as outlined in Table 2. If a dose reduction is required below the lowest Compound (10b) dose previously tested, the dose should be selected after discussion between the investigator and Navire's medical monitor.

TABLE 3
Dose Adjustments of Compound (10b) for Drug-Related Adverse Events

| Grade | Occurrence | Dose Modification^{a} |
|---|---|---|
| 1 or 2 | Any time | No dose reduction. |
| 3 | 1st and | Hold treatment with Compound (10b). |
| | 2nd time | If the drug-related AE resolves to ≤Grade 1 or baseline ≤14 days, |
| | | resume Compound (10b) at a lower dose level that was cleared by the |
| | | SRC. Dose re-escalation to the dose of Compound (10b) at which the |
| | | drug-related AE occurred, is not permitted. |
| | | If the drug-related AE persists for >14 days, discontinue patient from |
| | | Compound (10b) permanently |
| | 3rd time | Discontinue patient from Compound (10b) permanently. |
| | | (For patients with clinical benefit/response from Compound (10b) |
| | | continuation may be considered if toxicities resolve to ≤Grade 1 and with |
| | | proper dose adjustments after consultation between the investigator and |
| | | the medical monitor). |
| 4 | Any time | Discontinue patient from Compound (10b) permanently. |
| | | (For patients with clinical benefit/response from Compound (10b) |
| | | continuation may be considered if toxicities resolve to ≤Grade 1 and with |
| | | proper dose adjustments after consultation between the investigator and |
| | | the medical monitor). |

Abbreviations:
ADR = adverse drug reaction
^{a}These general guidelines constitute guidance to the investigator and may be supplemented by discussions with the medical monitor in specific cases.

### B. Other Allowable Compound (10b) Dose Modifications

Treatment interruptions and dose modifications other than the ones mentioned above can be considered for Compound (10b) after discussion between the investigator and Navire's medical monitor and proper documentation of the rationale.

Any changes should be within the following guidelines:
Further dose reductions of Compound (10b) can be made to keep clinically significant drug-related AEs Grade 52. Dose adjustments by more than 1 dose level at a time can be considered when judged in the best interest of the patient (e.g., severe myelosuppression, lactic acidosis) when the toxicity has resolved.
If a Grade 3 AE considered related to Compound (10b) is observed, treatment may be delayed for up to 2 weeks to allow resolution of the toxicity, with re-treatment at a reduced dose at the discretion of the investigator. If Grade 3 nausea, emesis, diarrhea, or clinically significant electrolyte abnormalities occur and resolve within 3 days on optimum treatment, treatment may be resumed without dose reduction. If recurrent Grade 3 nausea, emesis, diarrhea, or clinically significant electrolyte abnormalities occur of >3 days occur, treatment can be resumed with dose reduction after resolution of symptoms.
If a patient experiences more than one AE, Compound (10b) dose decisions will be based on the most severe AE.
A patient who requires a dose reduction and subsequently experiences a recurrent, clinically significant drug-related AE may undergo one additional dose reduction. Patients who continue to experience the event after two dose reductions will be withdrawn from the study. The lower doses will be based first on the lower doses cleared by the SRC as part of the dose escalation of this study and if lower doses are required, based on SRC cleared doses in Study NAV-1001.

### C. Nivolumab

Nivolumab may be interrupted, delayed, or discontinued depending on how well the patient tolerates the treatment. Nivolumab dosing decisions should be based on the CTCAE grading scale of drug-related AEs according to Table 4. Delay nivolumab dosing for any AE, laboratory abnormality, or intercurrent illness which, in the judgment of the investigator, warrants delaying the dose of study treatment. Patients may resume treatment with nivolumab if they have completed AE management (i.e., corticosteroid taper) or are on ≤10 mg prednisone or equivalent and meet the requirements per Table 4.

There will be no intra-patient dose escalations or reductions of nivolumab allowed. Patients may be dosed no less than 25 days from the previous dose. Pre-medications are not recommended for the first dose of nivolumab. Patients should be carefully monitored for acute infusion-related reactions (IRRs) during nivolumab administration. If an acute IRR is noted, manage the patient as described in Section 6.1.6 of the clinical protocol.

TABLE 4
Drug-Related Adverse Event Criteria for Delay,
Resume, and Discontinue of Nivolumab

| | | | |
|---|---|---|---|
| Drug-Related AE per | | Action | Clarifications, Exceptions, |
| CTCAE v5 | Severity | Taken | and Resume Criteria |

GASTROINTESTINAL

| | | | |
|---|---|---|---|
| Colitis or Diarrhea | Grade 2 | Delay | Dosing may resume when AE |
| | or 3 | dose | resolves to baseline |
| | Grade 4 | Permanently | |
| | | discontinue | |

RENAL

| | | | |
|---|---|---|---|
| Serum Creatinine | Grade 2 | Delay | Dosing may resume when AE |
| Increased | or 3 | dose | resolves to Grade ≤1 or |
| | | | baseline value |
| | Grade 4 | Permanently | |
| | | discontinue | |

PULMONARY

| | | | |
|---|---|---|---|
| Pneumonitis | Grade 2 | Delay | Dosing may resume after |
| | | dose | pneumonitis has resolved |
| | | | to ≤Grade 1. |
| | Grade 3 | Permanently | |
| | or 4 | discontinue | |

HEPATIC

| | | | |
|---|---|---|---|
| AST, ALT, or | AST or ALT > | Delay | Dosing may resume when |
| TBL increased | 3× and ≤5 × ULN | dose | laboratory values return |
| | or TBL > | | to baseline. |
| | 1.5× and | | |
| | ≤3 × ULN, | | |
| | regardless of | | |
| | baseline value | | |
| | AST or ALT > | Permanently | |
| | 5 × ULN or | discontinue | |
| | TBL > 3 × ULN, | | |
| | regardless of | | |
| | baseline value | | |
| | Concurrent AST | Permanently | |
| | or ALT > 3 × | discontinue | |
| | ULN and TBL > | | |
| | 2 × ULN, | | |
| | regardless of | | |
| | baseline value | | |

ENDOCRINOPATHY

| | | | |
|---|---|---|---|
| Adrenal Insufficiency | Grade 2 | Delay | Dosing may resume after |
| | adrenal | dose | adequately controlled with |
| | insufficiency | | hormone replacement. |
| | Grade 3 | Delay | Mandatory discussion with |
| | or 4 | dose or | and approval from the |
| | adrenal | permanently | medical monitor needed prior |
| | insufficiency or | discontinue | to resuming therapy. If |
| | adrenal crisis | | adrenal insufficiency resolves |
| | | | or is adequately controlled |
| | | | with physiologic hormone |
| | | | replacement, patient may not |
| | | | require discontinuation of |
| | | | study drug. |
| Hyperglycemia | Hyperglycemia | Delay | Dosing may resume if |
| | requiring | dose | hyperglycemia resolves to |
| | initiation or | | Grade ≤1 or baseline value, |
| | change in daily | | or is adequately controlled |
| | management | | with glucose-controlling |
| | (Grade 2 | | agents. |
| | or 3) | | |
| | Grade 4 | Permanently | |
| | | discontinue | |
| Hypophysitis/ | Symptomatic | Delay | Dosing may resume if |
| Hypopituitarism | Grade 1-3 that is | dose | endocrinopathy resolves to be |
| | also associated | | asymptomatic, or is |
| | with | | adequately controlled with |
| | corresponding | | only physiologic hormone |
| | abnormal lab | | replacement. |
| | and/or pituitary | | |
| | scan | | |
| | Grade 4 | Permanently | |
| | | discontinue | |
| Hyperthyroidism or | Grade 2 | Delay | Dosing may resume if |
| Hypothyroidism | or 3 | dose | endocrinopathy resolves to |
| | | | be asymptomatic, or is |
| | | | adequately controlled with |
| | | | only physiologic hormone |
| | | | replacement or other medical |
| | | | management. |
| | Grade 4 | Permanently | |
| | | discontinue | |

SKIN

| | | | |
|---|---|---|---|
| Rash | Grade 2 rash | Delay | Dosing may resume when |
| | covering >30% | dose | rash reduces to ≤10% body |
| | body surface | | surface area |
| | area or Grade 3 | | |
| | rash | | |
| | Suspected | Delay | Dosing may resume if SJS, |
| | Stevens-Johnson | dose | TEN, or DRESS is ruled out |
| | syndrome (SJS), | | and rash reduces to ≤10% |
| | toxic epidermal | | body surface area |
| | necrolysis (TEN) | | |
| | or drug reaction | | |
| | with eosinophilia | | |
| | and systemic | | |
| | symptoms | | |
| | (DRESS) | | |
| | Grade 4 rash or | Permanently | |
| | confirmed SJS, | discontinue | |
| | TEN, or DRESS | | |

NEUROLOGICAL

| | | | |
|---|---|---|---|
| Guillain-Barre | Any Grade | Permanently | |
| Syndrome (GBS) | | discontinue | |
| Myasthenia | Any Grade | Permanently | |
| Gravis (MG) | | discontinue | |
| Encephalitis | Any grade | Delay | After workup for differential |
| | encephalitis | dose | diagnosis, (i.e., infection, |
| | | | tumor-related), if encephalitis |
| | | | is not drug related, then |
| | | | dosing may resume when AE |
| | | | resolves |
| | Any grade drug- | Permanently | |
| | related | discontinue | |
| | encephalitis | | |
| Myelitis | Any grade | Delay | After workup for differential |
| | myelitis | dose | diagnosis, (i.e., infection, |
| | | | tumor-related), if myelitis is |
| | | | not drug related, then dosing |
| | | | may resume when AE |
| | | | resolves |
| | Any grade drug- | Permanently | |
| | related myelitis | discontinue | |
| Neurological (other than | Grade 2 | Delay | Dosing may resume when AE |
| GBS, MG, encephalitis, | | dose | resolves to baseline |
| or myelitis) | | | |
| | Grade 3 | Permanently | |
| | or 4 | discontinue | |

MYOCARDITIS

| | | |
|---|---|---|
| Symptoms | Delay | Dosing may resume after |
| induced from | dose | myocarditis has resolved |
| mild to moderate | | |
| activity or | | |
| exertion | | |
| Severe or life | Permanently | |
| threatening, with | discontinue | |
| symptoms at rest | | |
| or with minimal | | |
| activity or | | |
| exertion, and/or | | |
| where | | |
| intervention | | |
| indicated. | | |

OTHER CLINICAL

| | | | |
|---|---|---|---|
| Pancreatitis: Amylase | Grade 3 with | Delay | Note: Grade 3 increased |
| or Lipase increased | symptoms | dose | amylase or lipase without |
| | | | signs or symptoms of |
| | | | pancreatitis does not require |
| | | | dose delay. Dosing may |
| | | | resume when patient becomes |
| | | | asymptomatic. |
| | Grade 4 | Permanently | |
| | | discontinue | |
| Uveitis | Grade 2 | Delay | Dosing may resume if uveitis |
| | | dose | responds to topical therapy |
| | | | (eye drops) and after uveitis |
| | | | resolves to Grade ≤1 or |
| | | | baseline. If patient requires |
| | | | oral steroids for uveitis, then |
| | | | permanently discontinue |
| | | | study drug. |
| | Grade 3 | Permanently | |
| | or 4 | discontinue | |
| Hypersensitivity | Grade 3 | Permanently | Manifested by fever, chills, |
| reaction or IRR | or 4 | discontinue | rigors, headache, rash, |
| | | | pruritus, arthralgia, |
| | | | hypotension, hypertension, |
| | | | bronchospasm, or other |
| | | | allergic-like reactions. |
| | | | Refer to Section 6.1.6 |
| | | | of the clinical protocol |
| | | | on treatment of IRRs |
| Other Drug-related AE | Grade 2 non-skin | Delay | Dosing may resume when AE |
| (not listed above) | AE, except | dose | resolves to Grade ≤1 or |
| | fatigue | | baseline value. |
| | Grade 3 - First | Delay | Dosing may resume when AE |
| | occurrence | dose | resolves to Grade ≤1 or |
| | lasting ≤7 days | | baseline value. |
| | Grade 3 - First | Permanently | |
| | occurrence | discontinue | |
| | lasting >7 days | | |
| | Grade 3 - | Permanently | |
| | Reoccurence of | discontinue | |
| | any duration | | |
| | Grade 4 or life- | Permanently | |
| | threatening | discontinue | |

OTHER LAB ABNORMALITIES

| | | | |
|---|---|---|---|
| Any | Grade 3 | Delay | Exceptions: |
| | | dose | No delay required for |
| | | | Grade 3 lymphopenia |
| | | | Permanent |
| | | | Discontinuation for: |
| | | | Grade 3 |
| | | | thrombocytopenia >7 |
| | | | days or associated |
| | | | with bleeding. |
| Any | Grade 4 | Permanently | Exceptions: The following |
| | | discontinue | events do not require |
| | | | discontinuation of study drug: |
| | | | Grade 4 neutropenia ≤7 |
| | | | days |
| | | | Grade 4 lymphopenia |
| | | | or leukopenia |
| | | | Grade 4 isolated electrolyte |
| | | | imbalances/abnormalities that |
| | | | are not associated with |
| | | | clinical sequelae and are |
| | | | responding to |
| | | | supplementation/appropriate |
| | | | management within 72 hours |
| | | | of their onset |

Abbreviations:
ALT = alanine aminotransferase,
AST = aspartate aminotransferase,
CTCAE = Common Terminology Criteria for Adverse Events;
DRESS = Drug Rash with Eosinophilia and Systemic Symptoms,
SJS = Stevens Johnson Syndrome,
TBL = total bilirubin;
TEN = toxic epidermal necrolysis,
ULN = upper limit normal
Note:
Grading is based on the CTCAE v5.0

### Example 4: A Phase 1 Study of the SHP2 Inhibitor Compound (0b) in Combination with Nivolumab in Patients with Advanced Non-Small Cell Lung Cancer with a KRAS Mutation—Pharmacokinetics (PK) and Pharmacodynamics (PD)

a. Pharmacokinetics

Blood samples for PK evaluation of Compound (10b) and PK/ADA evaluation of nivolumab will be collected from all enrolled patients participating in the study. Time points of blood sample collection for PK of Compound (10b) are outlined in Table 5. Time points of blood sample collection of PK and ADA samples for nivolumab are outlined in Table 6. Additional details on collection and storage of samples can be found in the Laboratory Manual.

TABLE 5
Compound (10b) PK Sample Collection Schedule

| | | Time Points | | | | | |
|---|---|---|---|---|---|---|---|
| | | Pre-dose | | | | | |
| | | (within 2 hr | 0.5 hour | 1 hour | 2 hour | 4 hour | 6 hour |
| Cycle | | prior to | post-dose | post-dose | post-dose | post-dose | post-dose |
| Day | | Comp. (10b) dosing) | (±5 min) | (±10 min) | (±15 min) | (±15 min) | (±15 min) |
| 1 | 1 | X | X | X | X | X | X |
| 1 | 2 | X^{A} | | | | | |
| 1 | 8 | X | | | | | |
| 1 | 15 | X | | | | | |
| 2 | 1 | X | X | X | X | X | X |
| ≥3 | 1 | X | | | X | | |

^{A}Collect pre-dose (i.e., approximately 24 hr [±2 hr] after prior day's administration of Compound (10b) but before dosing of Compound (10b) on day of visit).

TABLE 6
Nivolumab PK and ADA Sample Collection Schedule

| | | PK Time Points | | |
|---|---|---|---|---|
| Cycle | | | End of Infusion (EOI) | ADA Time Points |
| Day | | Pre-dose^{A} | (within 5 min prior to EOI) | Pre-dose^{A} |
| 1 | 1 | X | X | X |
| 2 | 1 | X | X | X |
| ≥4^{B} | 1 | X | | X |

^{A}Pre-dose = within 2 hr prior to dosing of either Compound (10b) or nivolumab if Compound (10b) dosing is on hold
^{B}Collect blood samples pre-dose at C4D1 and then every 4 cycles thereafter (e.g., C8D1, C12D1)

On the days of PK sampling, patients should take the study drug(s) at the clinic as instructed by research staff Patients who forget and take their medication at home will be excluded from PK analysis for that day; they should not have blood samples collected. Complete dosing information, including the date and time of actual blood draw and time of the last study treatment dose prior to the sampling, should be obtained on all sampling days and recorded on the appropriate blood collection CRF. If any of the scheduled sampling times are missed or a sample is not drawn according to this schedule, the actual collection date and time will be recorded, and the remaining samples will be collected on schedule whenever possible.

Steady-state (C2D1 and beyond) PK samples should be collected after at least 7 consecutive days of dosing of Compound (10b).

If vomiting occurs within 4 hours following Compound (10b) administration on the day of PK blood sampling, the time (using a 24-hour clock) of vomiting should be recorded. No Compound (10b) should be taken in an effort to replace the material that has been vomited. The occurrence and frequency of any vomiting during a treatment cycle must be noted in the AE CRF. PK collection should continue as scheduled in all cohorts.

### B. Pharmacodynamics

Blood and tumor samples will be used to assess the pharmacodynamic effects of Compound (10b) in combination with nivolumab and potential mechanisms of antitumor response and resistance including but not limited to assessments provided in Table 7. These samples will also be used to develop exploratory biomarkers that may be used to predict response or resistance to Compound (10b) in combination with nivolumab.

Fresh tumor biopsies should be collected for all patients enrolled in the study if at all feasible. If fresh tumor biopsies were not done during molecular prescreening and are not feasible during Screening, the most recent archival tumor biopsies, collected preferably within 6 months but no later than 1 year of enrollment, with sufficient tumor for central testing of KRAS mutations can be used. Fine needle aspirates or other cytology samples are not acceptable.

Patients may be enrolled even when tumor biopsies cannot be obtained at the discretion of the medical monitor, in consultation with the investigator. Enrollment of patients for whom collection of tumor biopsies will not occur will be handled on a case-by-case basis by the medical monitor.

Refer to the Laboratory Manual for additional details regarding collection and handling of blood and tumor tissue samples.

The samples obtained will be used in different biomarker studies, including:
Gene expression within biopsied tumor tissue and circulating in the blood to identify genes or a signature of genes whose expression is modulated by Compound (10b), and which may serve as pharmacodynamic biomarkers.
Molecular profiling (e.g., NGS, PCR, etc) of tumor biopsies and ctDNA (liquid biopsies) to evaluate potential mechanisms of drug resistance by the above assays.
pSHP2 and pERK assessment by immunohistochemistry (IHC) in tumor biopsy as a measure of target engagement and MAPK pathway flux.
pERK assessment by multiparameter flow cytometry in blood PBMCs as a measure of MAPK pathway flux.
Tumor microenvironment (TME) profiling in tumor biopsies via IHC or multiplex immunofluorescence as a measure of tumor immune cell infiltration and activation, including baseline assessment of PD-L1.

TABLE 7
Pharmacodynamic Assessments

| Screening | Cycle 1 | | Cycle 2 | Subsequent Cycles | EOT V1 |
|---|---|---|---|---|---|
| Study Day | | | | | |
| −30 to −1 | 1 | 2 | 1 | 1 | Last dose +28 days |
| +/−days allowable | | | | | |
| 0 | 0 | 0 | ±3 | ±3 | ±3 |

Tumor biopsy

| | | | |
|---|---|---|---|
| PD-L1 (28-8) IHC | X | | |
| pSHP2 IHC | X | X | X |
| pERK IHC | X | X | X |
| TME biomarkers IHC/IF | X | X | X |
| Transcriptomics | X | X | X |

Blood

| | | | | | | |
|---|---|---|---|---|---|---|
| PBMC pERK | | X^{A} | X^{B} | X^{A} | X^{C} | X |
| ctDNA NGS | X | | | X | X^{D} | X |

^{A}Collect pre-dose (within 2 hr prior to dosing of Compound (10b) or nivolumab if Compound (10b) dosing is on hold) and 2 (±15 min) and 4 hr (±15 min) after administration of Compound (10b) (or nivolumab if Compound (10b) dosing is on hold).
^{B}Collect pre-dose (i.e., approximately 24 hr [±2 hr] after prior day's administration of Compound (10b) but before dosing of Compound (10b) on day of visit)
^{C}Collect pre-dose within 2 hr prior to and 2 hr (±15 min) after administration of Compound (10b) on day of the visit. Samples should be collected after at least 7 consecutive days of Compound (10b) dosing
^{D}Samples should be collected after at least 7 consecutive days of Compound (10b) dosing.

### Example 5: A Phase 1 Study of the SHP2 Inhibitor Compound (10b) in Combination with Nivolumab in Patients with Advanced Non-Small Cell Lung Cancer with a KRAS Mutation—BOIN Design

A BOIN design with target toxicity rate of ϕ=0.25 and a maximum sample size of 21 will be used to find the Compound (10b) MTD. As shown in FIG. 3, the BOIN design uses the following rule, optimized to minimize the probability of incorrect dose assignment, to guide dose escalation/de-escalation:
if the observed DLT rate at the current dose is ≤0.197, escalate the dose to the next higher dose level;
if it is ≥0.298, de-escalate the dose to the next lower dose level;
otherwise, stay at the current dose.

For the purpose of overdose control, doses j and higher levels will be eliminated from further examination if Pr (pⱼ>0.25|data) >0.95, where pⱼ is the true DLT rate of dose level j, j=1, . . . , 6. This posterior probability is evaluated based on the beta-binomial model yⱼ|pⱼ˜binomial(pⱼ) with pⱼ˜uniform(0, 1), where yⱼ is the number of patients experienced DLT at dose level j. When the lowest dose is eliminated, stop the trial for safety. The probability cutoff 0.95 is chosen to be consistent with the common practice that when the target DLT rate ϕ≤⅙, a dose with ⅔ patients experiencing DLT is eliminated.

The steps to implement the BOIN design are described as follows:
1. To assign a dose to the next cohort of patients (which will be done by the SRC), dose escalation/de-escalation will be conducted according to the rules displayed in Table 8 and based on a target toxicity rate of 25%. When using Table 8, please note the following:
   a. “Eliminate” means eliminate the current and higher doses from the trial to prevent treating any future patients at these doses because they are overly toxic.
   b. When a dose is eliminated, automatically de-escalate the dose to the next lower level. When the lowest dose is eliminated, the trial will be stopped for safety. In this case, no dose should be selected as the MTD.
   c. If none of the actions (i.e., escalation, de-escalation, or elimination) are triggered, new patients will be treated at the current dose.
   d. If the current dose is the lowest dose and the rule indicates dose de-escalation, new patients will be treated at the lowest dose unless the number of DLTs reaches the elimination boundary, at which point the trial will be terminated for safety.
   e. If the current dose is the highest dose and the rule indicates dose escalation, new patients will be treated at the highest dose.
   f. In the event that operational/practical circumstances result in an over-enrollment (i.e., the current cohort size >3), the next dose level decision would be based on the actual number of patients exposed in the cohort.
2. Dose escalation will start with 5-7 patients being enrolled at Dose level—2 (i.e., the lowest dose level). Per Table 8, if one DLT is observed in any of 6 patients enrolled, escalate to the next dose level. If a DLT is observed in ≥2 of 6 patients enrolled, then de-escalate to a lower dose.
3. If escalated to a higher dose level, follow same approach as in 2.
4. The maximum sample size for dose escalation is 21 or 10 in any cohort.

TABLE 8
Dose Escalation Decision Rules

| | The number of patients treated at the current dose | | | | | | |
|---|---|---|---|---|---|---|---|
| Actions ^{a} | 5 | 6 | 7 | 8 | 9 | 10 | 11 |
| Escalate if # of patients with DLT≤ | 0 | 1 | 1 | 1 | 1 | 1 | 2 |
| De-escalate if # of patients with DLT≥ | 2 | 2 | 3 | 3 | 3 | 3 | 4 |
| Eliminate if # of patients with DLT≥ | 3 | 4 | 4 | 5 | 5 | 5 | 6 |

Abbreviations: DLT = dose limiting toxicity
^{a} When none of the actions (i.e., escalate, de-escalate, or eliminate) are triggered, stay at the current dose for treating the next cohort of patients.
Note that “# of DLT” is the number of patients with at least 1 DLT.

After the trial is completed, select the MTD based on isotonic regression as specified in Liu and Yuan (2015). This computation is implemented by the “Estimate MTD” tab of the BOIN Design Desktop Program. Specifically, select as the MTD the dose for which the isotonic estimate of the DLT rate is closest to the target DLT rate. If there are ties, select the higher dose level when the isotonic estimate is lower than the target DLT rate and select the lower dose level when the isotonic estimate is greater than or equal to the target DLT rate. The RP2D for cohort expansion will be chosen based on toxicity (i.e., MTD), as well as other clinical considerations, e.g., PK/pharmacodynamics.

Table 9 shows the operating characteristics of the trial design based on 1000 simulations of the trial using the BOIN Design Desktop Program (MD Anderson Cancer Center, 2021). The operating characteristics show that the design selects the true MTD, if any, with high probability and allocates more patients to the dose levels with the DLT rate closest to the target of 0.3.

TABLE 9
Operating Characteristics for BOIN Dose Finding from Simulation

| | Dose Level | | | | | | Number of | % Early |
|---|---|---|---|---|---|---|---|---|
| | 1 | 2 | 3 | 4 | 5 | 6 | Patients | Stopping |
| Scenario 1 | | | | | | | | |
| True DLT Rate | 0.11 | 0.27 | 0.41 | 0.48 | 0.55 | 0.62 | | |
| Selection % | 18.4 | 47.4 | 22.8 | 8.9 | 1.8 | 0.1 | | 0.6 |
| % Pts Treated | 21.5 | 35.1 | 23.0 | 12.4 | 5.7 | 2.3 | 19.8 | |
| Scenario 2 | | | | | | | | |
| True DLT Rate | 0.06 | 0.13 | 0.27 | 0.42 | 0.51 | 0.61 | | |
| Selection % | 1.9 | 21.0 | 46.7 | 23.4 | 6.7 | 0.2 | | 0.1 |
| % Pts Treated | 8.8 | 21.7 | 32.6 | 22.5 | 10.2 | 4.2 | 20.3 | |
| Scenario 3 | | | | | | | | |
| True DLT Rate | 0.04 | 0.08 | 0.11 | 0.27 | 0.44 | 0.56 | | |
| Selection % | 0.3 | 2.2 | 20.5 | 51.8 | 22.6 | 2.6 | | 0.0 |
| % Pts Treated | 7.0 | 10.1 | 20.1 | 30.8 | 22.7 | 9.5 | 20.5 | |
| Scenario 4 | | | | | | | | |
| True DLT Rate | 0.02 | 0.06 | 0.09 | 0.13 | 0.27 | 0.45 | | |
| Selection % | 0.0 | 0.8 | 4.4 | 23.4 | 54.2 | 17.2 | | 0.0 |
| % Pts Treated | 5.6 | 8.3 | 11.0 | 20.5 | 33.1 | 21.6 | 20.5 | |
| Scenario 5 | | | | | | | | |
| True DLT Rate | 0.03 | 0.06 | 0.08 | 0.11 | 0.13 | 0.27 | | |
| Selection % | 0.1 | 0.6 | 3.1 | 6.4 | 27.0 | 62.8 | | 0.0 |
| % Pts Treated | 6.3 | 8.7 | 10.5 | 12.6 | 21.2 | 40.6 | 19.8 | |

Abbreviations:
BOIN = Bayesian optimal interval design;
DLT = dose limiting toxicity
Note:
“% Early Stopping” refers to early stopping due to excessive DLT.

### Example 6: A Phase 1 Study of the SHP2 Inhibitor Compound (10b) in Combination with Nivolumab in Patients with Advanced Non-Small Cell Lung Cancer with a KRAS Mutation—Efficacy

Although response is not the primary endpoint of this trial, patients will be assessed by standard criteria (Note: all patients must have measurable disease at study entry and therefore will be assessed per RECIST v1.1 criteria). For the purposes of this study, tumor response should be assessed every 8 weeks (±7 days) (i.e., Day 1 of every odd cycle) thereafter until PD. In addition to a baseline scan, confirmatory scans will also be obtained at least 4 weeks following initial documentation of an objective response (i.e., PR or CR).

Response and progression will be evaluated in this study using RECIST v1.1; the new international criteria proposed by the RECIST Committee (Eisenhauser, 2009). Changes in only the largest diameter (unidimensional measurement) of the tumor lesions are used in the RECIST criteria.

### A. Definitions

Evaluable for safety. All patients will be evaluable for safety from the time of their first treatment with either study medication. Only patients who received ≥75% or more of planned Compound (10b) doses and nivolumab during Cycle 1 are evaluable for DLT assessment, unless the patients stopped therapy due to toxicity.

Evaluable for objective response. Only those patients who have measurable disease present at baseline, have received at least one cycle of therapy, and have had their disease re-evaluated will be considered evaluable for response. These patients will have their response classified according to the definitions stated below. (Note: Patients who exhibit objective disease progression prior to the end of Cycle 1 will also be considered evaluable.)

### B. Disease Parameters

Measurable disease. Measurable lesions are defined as those that can be accurately measured in at least one dimension (longest diameter [LD] to be recorded) as ≥10 mm by scan (CT, MRI, or PET-CT; scan slice thickness should be no greater than 5 mm). All tumor measurements must be recorded in millimeters (or decimal fractions of centimeters).

Non-measurable disease. All other lesions (or sites of disease), including small lesions (LD<10 mm or pathological lymph nodes with ≥10 mm to <15 mm) are considered non-measurable disease. Bone lesions, leptomeningeal disease, ascites, pleural/pericardial effusions, lymphangitis cutis/pulmonis, inflammatory breast disease, abdominal masses (not followed by CT, MRI, or PET-CT), and cystic lesions are all truly non-measurable.

Target lesions. All measurable lesions up to a maximum of 5 lesions per organ and 10 lesions in total, representative of all involved organs, should be identified as target lesions and recorded and measured at baseline. Target lesions should be selected on the basis of their size (lesions with the LD) and their suitability for accurate repeated measurements (either by imaging techniques or clinically). A sum of the LD for all target lesions will be calculated and reported as the baseline sum LD. The baseline sum LD will be used as reference by which to characterize the objective tumor response.

Non-target lesions. All other lesions (or sites of disease) including any measurable lesions over and above the 10 target lesions should be identified as non-target lesions and should also be recorded at baseline. Measurements of these lesions are not required, but the presence or absence of each should be noted throughout follow-up.

### C. Methods for Evaluation of Measurable Disease

All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination when both methods have been used to assess the antitumor effect of a treatment.

CT, MRI, or PET-CT scans should be used to measure lesions selected for response assessment: These techniques should be performed with cuts of 5 mm or less in slice thickness contiguously. The same method of assessment and the same technique should be used to characterize each identified and reported lesion at baseline and during follow-up.

Tumor markers: Tumor markers alone cannot be used to assess response. If markers are initially above the upper normal limit, they must normalize for a patient to be considered in complete clinical response. Specific additional criteria for standardized usage of prostate-specific antigen and CA-125 response in support of clinical trials are being developed.

Cytology, Histology: These techniques can be used to differentiate between PR and CR in rare cases (e.g., residual lesions in tumor types, such as germ cell tumors, where known residual benign tumors can remain).

The cytological confirmation of the neoplastic origin of any effusion that appears or worsens during treatment when the measurable tumor has met criteria for response or stable disease is mandatory to differentiate between response or stable disease (an effusion may be a side effect of the treatment) and PD.

### D. Response Criteria

### Evaluation of Target Lesions

Complete Response (CR): Disappearance of all target lesions.

Partial Response (PR): At least a 30% decrease in the sum of the LD of target lesions, taking as reference the baseline sum LD.

Progressive Disease (PD): At least a 20% increase in the sum of the LD of target lesions, taking as reference the smallest sum LD recorded since the treatment started or the appearance of one or more new lesions.

Stable Disease (SD): Neither sufficient shrinkage to qualify for PR nor sufficient increase to qualify for PD, taking as reference the smallest sum LD since the treatment started.

### Evaluation of Non-Target Lesions

Complete Response (CR): Disappearance of all non-target lesions and normalization of tumor marker level.

Note: If tumor markers are initially above the upper normal limit, they must normalize for a patient to be considered in complete clinical response.

Stable Disease (SD): Persistence of one or more non-target lesion(s) and/or maintenance of tumor marker level above the normal limits

Progressive Disease (PD): Appearance of one or more new lesions and/or unequivocal progression of existing non-target lesions

Although a clear progression of “non-target” lesions only is exceptional, the opinion of the treating physician should prevail in such circumstances, and the progression status should be confirmed at a later time by the review panel (or principal investigator).

### Evaluation of Best Overall Response

The best overall response is the best response recorded from the start of the treatment until disease progression/recurrence (taking as reference for PD the smallest measurements recorded since the treatment started). The patient's best response assignment will depend on the achievement of both measurement and confirmation criteria.

TABLE 10
Evaluation of Best Overall Response

| Target | Non-Target | New | Overall | Best Response for this |
|---|---|---|---|---|
| Lesions | Lesions | Lesions | Response | Category Also Requires: |
| CR | CR | No | CR | ≥4 wk confirmation |
| CR | Non-CR/Non-PD | No | PR | ≥4 wk confirmation |
| PR | Non-PD | No | PR | |
| SD | Non-PD | No | SD | Documented at least once ≥4 |
| | | | | wk from baseline |
| PD | Any | Yes or No | PD | No prior SD, PR or CR |
| Any | PD^{a} | Yes or No | PD | |
| Any | Any | Yes | PD | |

^{a}In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration”. Every effort should be made to document the objective progression even after discontinuation of treatment.

### E. Duration of Response

Duration of overall response: DOR is measured from the time measurement criteria are met for CR or PR (whichever is first recorded) until the first date that recurrent or PD is objectively documented (taking as reference for PD the smallest measurements recorded since the treatment started).

The duration of overall CR is measured from the time measurement criteria are first met for CR until the first date that recurrent disease is objectively documented.

Duration of stable disease: Stable disease is measured from the start of the treatment until the criteria for progression are met, taking as reference the smallest measurements recorded since the treatment started.

### F. Progression Free Survival

Progression free survival (PFS) is defined as the date of the start of treatment to the date of the event defined as the first documented progression or death due to any cause. If patient has not had an event, PFS will be censored according to details described in the SAP. Kaplan-Meier analysis of PFS will be provided.

### Example 7: Combination of Formula (10b) and Anti-PD-1 in CT-26 CRISPR Knock-In KRAS D12C—In Vivo Efficacy

### a. Materials

Test article #1—formula (10b)

Test article #2—ANTIBODY A; Variable region epitope: Mouse PD-1; Constant region: Mouse Immunoglobulin G1 (IgG1); engineered with a single amino acid substitution, aspartic acid to alanine at codon 265 (D265A), which prevents Fcγ receptor binding; and Molecular weight: ˜150 kDa.

Isotype Control article #1—MOPC-21, InVivoMAb mouse IgG1 Isotype Control; Variable region epitope: unknown specificity; Constant region: Mouse Immunoglobulin G1 (IgG1); and Molecular weight: −150 kDa.

### B. Experimental Procedures

Animal Receipt and Housing. For the CT-26 CRISPR knock-in KRAS D12C study, protocols and procedures involving the care and use of animals were reviewed and approved by the Charles River Accelerator and Development Lab (CRADL) Institutional Animal Care and Use Committee (IACUC) prior to execution. The animal care and use program has been fully accredited by the Association for Assessment and Accreditation of Laboratory Animal Care International (AAALAC). Balb/c female mice (Envigo; Inotiv Inc.) were ordered, aged six to eight weeks at receipt, and identified by ear notch. Animals were acclimated for a period of eleven days prior to tumor inoculation. For the duration of the acclimation period and throughout the study, mice were group-housed up to five animals per cage in disposable polycarbonate IVC cages (Innovive; San Diego, CA) which consisted of a layer of Alpha-Dri bedding (Sheppard Specialty Papers, Inc., Kalamazoo, MI), a certified enrichment block and diamond-twist, and a certified polycarbonate housing device. Mice were presented with rodent diet pellet and irradiated reverse-osmosis Aquavive® drinking water (Innovive; San Diego, CA), ad libitum. The vivarium temperature was maintained between 20° C. through 26° C. and an environmental humidity between 30% through 70%. The interior room lighting was cycled twelve hours on and twelve hours off.

Tumor Cell Line Culture and Inoculation. The murine colon carcinoma CT-26 cell line was genetically engineered by CRISPR technology to edit the three endogenous loci of KRAS with D12C, and internally registered at BMS as BXA-229794-02-001. Cells were screened and confirmed negative for mouse pathogens (IDEXX) and maintained in-vitro at 37° C., 5% CO₂ atmosphere in 10% (v/v) fetal bovine serum (FBS, Invitrogen) and 90% (v/v) Roswell Park Memorial Institute (RPMI) 1640 Medium (Gibco) on T150 flasks. For implantation, cells were harvested with 0.25% Trypsin-EDTA (Gibco), washed in DPBS (Gibco), counted and viability determined with trypan blue exclusion using Vi-Cell XR (Beckman Coulter), then brought up in a 1:1 ratio of DPBS:Matrigel to a final concentration of 3×10⁶ cells/mL. The single-cell suspension was implanted into the subcutaneous right hind flank region of each mouse, 3×10⁵ cells, at a volume of 0.1 mL per mouse.

Group Assignment and Dosing. Following sixteen days post inoculation, Balb/c mice were assigned to a treatment group, with each group consisting of n=10 mice. A ‘stratified’ randomization method by tumor volume was selected (StudyLog® Desktop software, version 4.5.5.523) whereby group mean tumor volumes averaged about 191 mm³.

CT-26 KRAS D12C KI tumor-bearing female Balb/c mice (10 animals/group) were administered one of the following treatment regimens until reaching an experimental endpoint: 1) a combination of vehicle control (0.5% v/v methyl cellulose in sterile deionized water) (PO, QD×35) and MOPC-21 isotype control antibody (10 mg/kg PO, Q4D×4), 2) ANTIBODY A (10 mg/kg IP, Q4D×4), 3) formula (10b) (100 mg/kg PO, QD×35), 4) a combination of ANTIBODY A (10 mg/kg IP, Q4D×4) and formula (10b) (100 mg/kg PO, QD×35).

Formula (10b) Formulation. Formula (10b) formulation buffer (0.5% v/v methyl cellulose in sterile deionized water) was prepared once per month by weighing the desired amount of methyl cellulose 400 cp (e.g., Sigma-Aldrich, catalog M0262, viscosity 400 cP) into a glass bottle. Sterile deionized water equivalent to 75% v/v of the intended final volume was added under continuous magnetic stirring with a stir bar and stirred at room temperature until complete dissolution. The buffer was then brought to the final volume with sterile deionized water.

Formula (10b) working suspensions of 10 mg/mL of active pharmaceutical ingredient (10.22 mg/mL with correction factor) were prepared weekly and administered daily for up to 35 days at a 10 mL/kg dose volume by oral gavage to mice for the 100 mg/kg dose level, starting on Study Day 1. A correction factor of 1.022 was applied to the formula (10b) formulation to accommodate for the purity (97.8%). To prepare formula (10b) formulations, compound was accurately weighed into a glass vial. Formula (10b) formulation buffer equivalent to 70% v/v of the intended final volume was added to the glass vial containing drug substance and mixed well using a ¼-inch probe for 4 to 9 minutes until a homogeneous suspension was achieved with no large visible agglomerates or particles. The rest of the suspending vehicle was added to reach the intended final volume to the dispersion containing drug substance. The suspension was mixed well for 30 minutes and stored at 2 to 8° C. The suspension was well mixed prior to and throughout dosing each day.

ANTIBODY A Formulation. ANTIBODY A is a murine anti-PD-1 murine immunoglobulin G1-D265A (α-PD-1), stock concentration of 18.12 mg/mL. The mAb is engineered with a single amino acid substitution, aspartic acid to alanine at codon 265 (D265A), which prevents Fcγ receptor binding. Stock solution was stored in a monitored 2° C. through 8° C. refrigerator. Dosing solutions were prepared once per study to a 1 mg/mL concentration by dilution into PBS, pH 7.2, aliquoted and stored into sterile containers, and kept at 2° C. through 8° C. for the length of the dosing period. The 1 mg/mL ANTIBODY A formulation was administered every four days for four doses (Q4D×4), starting on Study Day 1, at 10 mL/kg dose volume by intraperitoneal to mice for a 10 mg/kg dose level.

MOPC-21 Isotype Control Antibody Formulation. MOPC-21 is a mouse IgG1 isotype control antibody, manufactured by BioXCell, lot 78512101 and stock concentration 10.22 mg/mL. The fragment antigen-binding site (Fab) size has unknown specificity to an antigen. Stock material was stored in a monitored 2° C. through 8° C. refrigerator. Dosing solutions were prepared to 1 mg/mL concentration by dilution into PBS, pH 7.2, aliquoted and stored into sterile containers, and kept at 2° C. through 8° C. for the length of the dosing period. The 1 mg/mL MOPC-21 formulation was administered every four days for four doses (Q4D×4), starting on Study Day 1, at 10 mL/kg dose volume by intraperitoneal to mice for a 10 mg/kg dose level.

Tumor and Body Weight Measurements. Tumor length [mm] and width [mm] were measured two times per week using a caliper, and the tumor volume [mm³] calculated using the formula: Tumor volume=(length×width²)/2, where length was defined as the longest tumor diameter and width was defined as the shortest tumor diameter.

Mouse body weights were recorded once weekly following randomization.

Calculations and Statistical Analyses. Median tumor volume and progression-free to tumor burden endpoint graphs, as well as median progression-free, hazard ratio and statistical analyses, were generated from GraphPad Prism software (version 9).

Median tumor volumes were calculated from each group to qualify efficacy of treatment prior to mice reaching tumor burden. If a mouse reached tumor burden in a particular group, the last recorded tumor volume was extended and included in later median calculations until the calculated median was greater than any single extended value, or until 50% or more of the mice in that group reached tumor burden. If a decedent was discovered prior to reaching an experimental endpoint, such as dosing error, individual tumor volume values from that subject were not included in the median for any timepoint.

A Kaplan-Meier plot was generated to evaluate the cumulative percentage to tumor burden endpoint of mice following treatment. A tumor burden endpoint was defined as a subject reaching an experimental endpoint of tumor burden with a recorded volume of 1,500 mm³ or greater. A hazard ratio, identifying the tumor burden incidence rate was calculated using the Mantel-Haenszel method.

A p-value of less than 0.05 was considered statistically significant following a Log-rank (Mantel-Cox) test comparing the combination formula (10b) plus ANTIBODY A against ANTIBODY A alone.

Experimental Endpoints. Following tumor cell inoculation, animals were checked daily for morbidity and mortality. During routine monitoring, animals were checked for any effects of tumor growth and treatments on behavior such as mobility, food and water consumption, body weight gain/loss, eye/hair matting and any other abnormalities. Mice were euthanized if they lost over 20% of their body weight relative to the weight on the first day of treatment. Additionally, mice were euthanized if significant adverse changes were noted to the animal's general welfare. A mouse was euthanized for tumor burden if the calculated tumor volume was ≥1,500 mm³. The study commenced once all mice reached tumor burden, determined as Study Day 35.

### C. Results

This study was performed to determine if the combination of the SHP2 inhibitor, formula (10b), and the murine anti-PD-1, ANTIBODY A, enhanced anti-tumor activity over either agent alone in vivo. The study was performed in the syngeneic CRC CT-26 model with a CRISPR engineered KRAS D12C knock-in mutation at all three endogenous loci.

Treatment with the combination of SHP2i, formula (10b), and anti-PD-1, ANTIBODY A, resulted in delayed progression to tumor burden in the subcutaneous CT-26 syngeneic model harboring KRAS D12C KI mutations. An in vivo study was conducted to evaluate the impact of formula (10b) in combination with the murine anti-PD-1 antibody, ANTIBODY A, on tumor volume in Balb/c mice bearing the cell line-derived CT-26 CRC syngeneic model, which harbored a KRAS D12C mutation at all three endogenous loci.

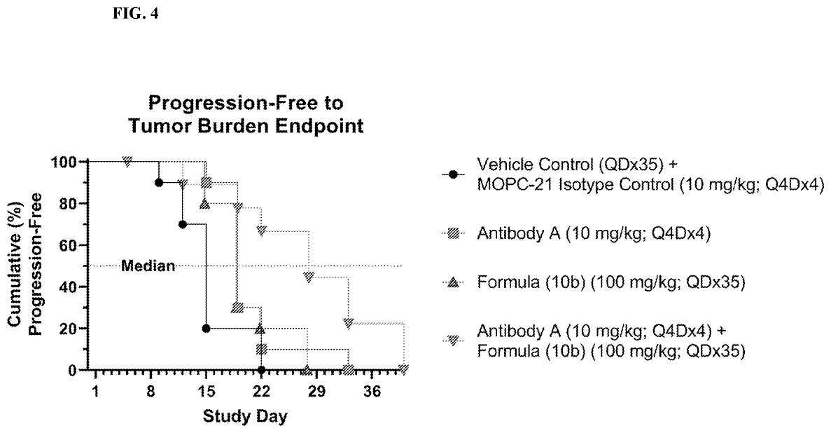

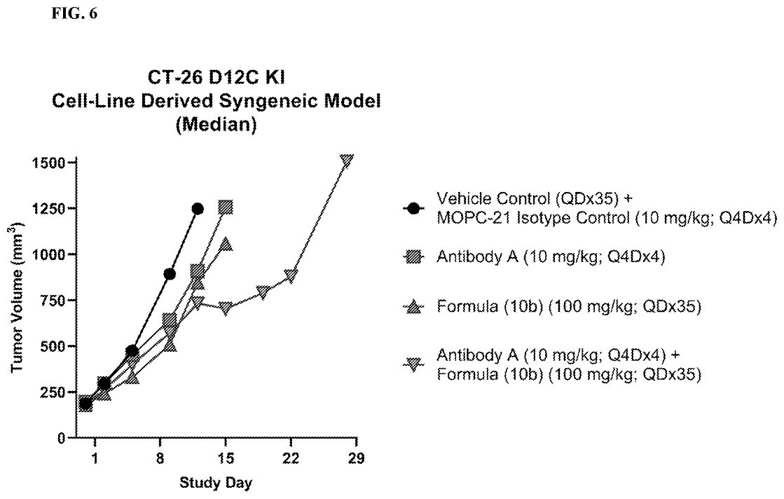

Following once daily oral administration of formula (10b) (100 mg/kg) for 35 days, or intraperitoneal administration of ANTIBODY A (10 mg/kg) every-four days for four doses, or in combination with formula (10b) (100 mg/kg) plus ANTIBODY A (10 mg/kg), a delay in progression to tumor burden was observed in the combination arm treated with formula (10b) and ANTIBODY A (FIG. 4 and FIG. 6). In FIG. 4: Tumor burden endpoint was defined where a subject was recorded with a tumor volume >1,500 mm³. Study Day 1 was defined as the first day of dosing.

As shown in Table 11, the median survival increased by nine days following administration of the combination formula (10b) plus ANTIBODY A compared to either agent alone or was increased by thirteen days when compared to the vehicle plus isotype control.

TABLE 11
Summary of Median Progression-Free to Tumor Burden Endpoint
in CT-26 Bearing Balb/c Mice with KRAS D12C KI Following
Administration of Vehicle plus Isotype Control, or Formula
(10b) and ANTIBODY A Alone or in Combination

| Group | | Dose Level | Median |
|---|---|---|---|
| (n = 10) | Treatment | (mg/kg) | (Study Day) |
| 1 | Vehicle Control+ | 0 | 15 |
| | MOPC-21 Isotype | 10 | |
| 2 | ANTIBODY A | 10 | 19 |
| 3 | Formula (10b) | 100 | 19 |
| 4 | ANTIBODY A+ | 10 | 28 |
| | Formula (10b) | 100 | |

Abbreviations: Balb/c = Bagg Albino; KRAS = Kirsten rat sarcoma viral oncogene homolog; D12C = aspartic acid to cysteine at amino acid codon number twelve; KI = Knock-In; n = number.
Note:
Tumor burden endpoint was defined where a subject was recorded with a tumor volume >1,500 mm³. Study Day 1 was defined as the first day of dosing.

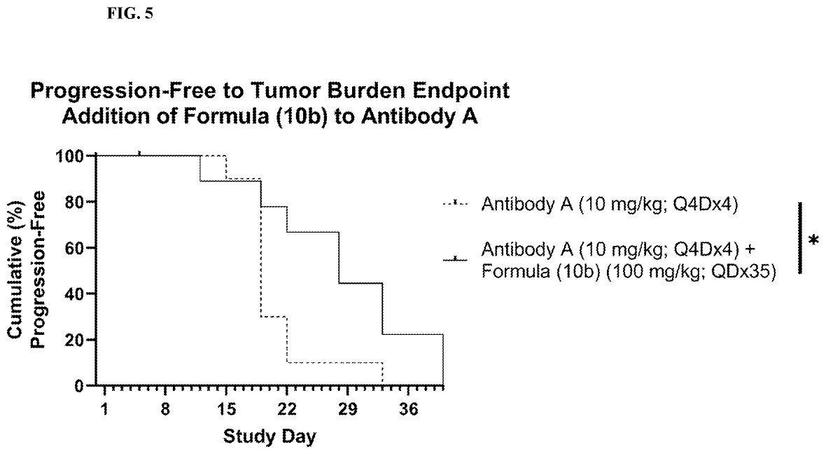

As shown in FIG. 5, a statistically significant delay in progression to tumor burden endpoint was observed, p=0.0309, when evaluating the benefit of adding the SHP2 inhibitor, formula (10b), to mice receiving the mouse surrogate for anti-PD-1 therapy, ANTIBODY A, from the two progression-free curves using the Log-rank (Mantel-Cox) test. In FIG. 5, Abbreviations: *, p<0.05. Comparison of progression-free curve ANTIBODY A versus combination with Formula (10b) performed with the Log-rank (Mantel-Cox) test. Tumor burden endpoint was defined where a subject was recorded with a tumor volume >1,500 mm³. Study Day 1 was defined as the first day of dosing.

In addition, Mantel-Haenszel hazard ratios were determined on each permutation between pairs of progression-free to tumor burden endpoint curves. In all comparisons, a reduced hazard ratio incidence rate was found in subjects receiving formula (10b) and ANTIBODY A in combination compared to those receiving either treatment alone, or to subjects receiving the vehicle plus isotype control (Table 12).

TABLE 12
Summary of the Hazard Ratio in CT-26 Bearing Balb/c Mice with
KRAS D12C KI Following Administration of Vehicle plus Isotype
Control, Formula (10b) and ANTIBODY A Alone or in Combination

| | | | Hazard Ratio | | |
|---|---|---|---|---|---|
| | | | Treatment/ | Treatment/ | Treatment/ |
| Group | | Dose Level | Control | ANTIBODY A | Formula (10b) |
| (n = 10) | Treatment | (mg/kg) | (Group 1) | (Group 2) | (Group 3) |
| 1 | Vehicle Control+ | 0 | n/a | — | — |
| | MOPC-21 Isotype | 10 | | | |
| 2 | ANTIBODY A | 100 | 0.244 | n/a | — |
| 3 | Formula (10b) | 10 | 0.261 | 1.230 | n/a |
| 4 | ANTIBODY A+ | 100 | 0.131 | 0.257 | 0.224 |
| | Formula (10b) | 10 | | | |

Abbreviations: Balb/c = Bagg Albino; KRAS = Kirsten rat sarcoma viral oncogene homolog; D12C = aspartic acid to cysteine at amino acid codon number twelve; KI = Knock-In; n = number; n/a = not applicable.
Note:
Hazard Ratio determined using the Mantel-Haenszel test based on progression-free to tumor burden endpoint curves. Tumor burden endpoint was defined where a subject was recorded with a tumor volume >1,500 mm³. Study Day 1 was defined as the first day of dosing.

### D. Conclusions

The result from this in vivo study demonstrates that the addition of SHP2 inhibitor, formula (10b), combined with a mouse surrogate for nivolumab, i.e., a murine anti-PD-1 antibody ANTIBODY A, showed a significant delay to tumor burden compared to anti-PD-1 treatment alone in the engineered KRAS D12C knock-in colorectal carcinoma CT-26 syngeneic model. The in-vivo study supports the clinical evaluation of the combination SHP2 inhibitor plus anti-PD-1 therapy, such as nivolumab, and shows that SHP2 inhibition by formula (10b) may potentiate the response of the anti-PD-1 antibody.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating cancer in a subject, comprising administering to the subject:
a) a therapeutically effective amount of a compound represented by formula (I): 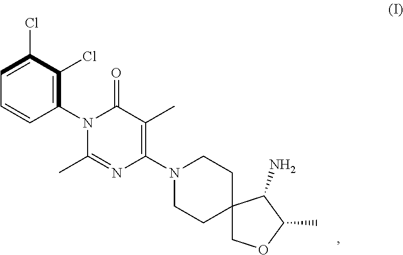
or a pharmaceutically acceptable salt, stereoisomer, conformational isomer, tautomer, or a combination thereof; and
b) a therapeutically effective amount of nivolumab.

2. The method of claim 1, wherein the compound of formula (I) is represented by formula (10b): 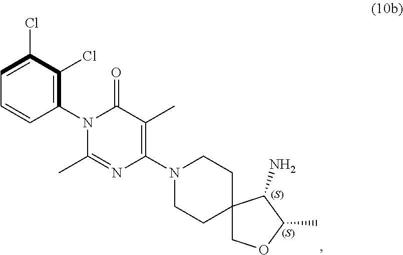
having the name of 6-((3S,4S)-4-amino-3-methyl-2-oxa-8-azaspiro[4.5]decan-8-yl)-3-(Rₐ)-(2,3-dichlorophenyl)-2,5-dimethyl-4(3H)-pyrimidinone.

3. The method of claim 1 or 2, wherein the cancer expresses PD-L1, the cancer displays a high level of microsatellite instability (MSI-H), a deficient mismatch repair (dMMR), or a high level of tumor mutational burden (TMB-H), or the cancer is a KRAS-positive cancer.

4. The method of any one of claims 1 to 3, wherein the cancer comprises a solid tumor and/or a liquid tumor.

5. The method of any one of claims 1 to 4, wherein the cancer is anal cancer, biliary tract cancer, bladder cancer, brain cancer, cervical cancer, colorectal cancer (CRC), endometrial cancer, esophageal cancer, gastric cancer, head and neck squamous cell carcinoma (HNSCC), hepatocellular carcinoma (HCC), merkel cell carcinoma, melanoma, mesothelioma, non-small cell lung cancer (NSCLC), ovarian cancer, prostate cancer, renal cell carcinoma (RCC), small cell lung cancer (SCLC), squamous cell carcinoma (SCC), triple negative breast cancer (TNBC), or a combination thereof.

6. The method of claim 5, wherein the cancer is colorectal cancer (CRC) or non-small cell lung cancer (NSCLC).

7. The method of claim 6, wherein the cancer is an advanced non-small cell lung cancer (NSCLC).

8. The method of any one of claims 1 to 7, wherein the cancer is resistant to a PD-1/PD-L1 inhibitor; and/or the cancer is characterized by intrinsic and/or acquired resistance to a PD-1/PD-L1 inhibitor.

9. The method of any one of claims 1 to 8, wherein the cancer is a PD-L-positive cancer resistant to pembrolizumab or nivolumab; and/or the cancer is a KRAS-positive cancer resistant to pembrolizumab or nivolumab.

10. The method of any one of claims 1 to 9, wherein the cancer is characterized by a KRAS mutation.

11. The method of any one of claims 1 to 10, wherein the cancer is characterized by a KRAS G12C mutation.

12. The method of any one of claims 1 to 11, wherein the cancer has progressed or recurred on or after at least one prior line of a systemic therapy comprising a platinum-based doublet chemotherapy and/or an anti-PD-1/PD-L1 therapy, each of which is given in monotherapy or both of which are given in combination therapy.

13. The method of any one of claims 1 to 12, wherein the cancer has progressed or recurred during a treatment of an anti-PD-1/PD-L1 therapy or within about 90 days after discontinuing an anti-PD-1/PD-L1 therapy.

14. The method of any one of claims 1 to 13, wherein the subject does not have an activating mutation in BRAF V600X, PTPN11 (SHP2), or KRAS Q61X.

15. The method of any one of claims 1 to 14, wherein the subject is not previously treated with a PTPN11 inhibitor, provided that the PTPN11 inhibitor is other than the compound of formula (I) or (10b).

16. The method of any one of claims 1 to 15, wherein the subject meets all of inclusion criteria of 1) to 10) according to Example 2, provided that the subject does not meet any one of exclusion criteria of 1) to 22) according to Example 2.

17. The method of any one of claims 1 to 16, wherein the subject is human.

18. The method of any one of claims 1 to 17, wherein the compound of formula (I) or (10b) and nivolumab are provided in jointly therapeutically effective amounts.

19. The method of any one of claims 1 to 17, wherein the compound of formula (I) or (10b) and nivolumab are provided in synergistically effective amounts.

20. The method of any one of claims 1 to 17, wherein the compound of formula (I) or (10b) and nivolumab are each used at a dose less than when each is used alone.

21. The method of any one of claims 1 to 20, wherein the treating comprises one or more treatment cycles; each of one or more treatment cycles has a duration of about 28 days; the compound of formula (I) or (10b) is administered daily; and nivolumab is administered every four (4) weeks.

22. The method of any one of claims 1 to 21, wherein the treating comprises a dose escalation period; and the administration of the compound of formula (I) or (10b) comprises one or more dose escalations, a dose retention, or a dose de-escalation, each of which is determined by a dose-limiting toxicity (DLT) assessment.

23. The method of claim 22, wherein the administration of the compound of formula (I) or (10b) comprises a dose escalation after a previous treatment cycle, when a dose-limiting toxicity (DLT) rate is less than about 19.7% determined by a DLT assessment.

24. The method of claim 22, wherein the administration of the compound of formula (I) or (10b) comprises a dose de-escalation after a previous treatment cycle, when a dose-limiting toxicity rate is more than about 29.8% determined by a DLT assessment.

25. The method of claim 22, wherein the administration of the compound of formula (I) or (10b) comprises a dose retention after a previous treatment cycle, when a dose-limiting toxicity rate is in a range of from about 21.9% to about 29.8% determined by a DLT assessment.

26. The method of any one of claims 22 to 25, wherein, after the dose escalation period, the treating further comprises a dose expansion period; and the compound of formula (I) or (10b) is administered at a dose regimen determined during the dose escalation period.

27. The method of claim 26, wherein, during the dose expansion period, the administration of the compound of formula (I) or (10b) optionally comprises one or more dose adjustments.

28. The method of any one of claims 1 to 27, wherein the therapeutically effective amount of nivolumab is a total dosage of about 480 mg every four (4) weeks.

29. The method of any one of claims 1 to 28, wherein the therapeutically effective amount of the compound of formula (I) or (10b) is a total daily dosage of from about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg, on a salt-free and anhydrous basis.

30. The method of claim 29, wherein the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis.

31. The method of claim 29 or 30, wherein the therapeutically effective amount is a total daily dosage of about 250 mg of the compound of formula (I) or (10b), on a salt-free and anhydrous basis.

32. The method of claim 29 or 30, wherein the therapeutically effective amount is a total daily dosage of about 400 mg of the compound of formula (I) or (10b), on a salt-free and anhydrous basis.

33. The method of claim 29 or 30, wherein the therapeutically effective amount is a total daily dosage of about 550 mg of the compound of formula (I) or (10b), on a salt-free and anhydrous basis.

34. The method of any one of claims 1 to 33, wherein the compound of formula (10b) is administered orally; and nivolumab is administered intravenously.

35. The method of any one of claims 1 to 34, wherein the compound of formula (10b) is administered once, twice, three times, or four times daily.

36. The method of claim 35, wherein the compound of formula (I) or (10b) is administered once daily; and nivolumab is administered once every four (4) weeks.

37. The method of any one of claims 1 to 36, wherein the treating reduces a volume of the cancer or a solid tumor at least about 10%, about 20%, about 30%, about 40%, about 50%, about 60%, about 70%, about 80%, or about 90%.

38. The method of any one of claims 1 to 37, wherein the treating stabilizes the cancer or a solid tumor.

39. The method of any one of claims 1 to 38, wherein the subject is further evaluated according to one or more parameters of Table 1 comprising plasma pharmacokinetic parameters and/or pharmacodynamic parameters.

40. The method of any one of claims 1 to 39, wherein the subject is further evaluated for one or more biomarkers that correlate to an antitumor response.

41. A kit for treating cancer in a subject, comprising:
a) a therapeutically effective amount of a compound represented by formula (I) according to claim 1 or a compound represented by formula (10b) according to claim 2; and
b) a therapeutically effective amount of nivolumab,
with instruction for effective administration.
